(19) United States
(12) Patent Application Publication
(10) Pub. No.: US 2024/0277798 A1
(43) Pub. Date: Aug. 22, 2024

(54) CYCLOSPORINE COMPOSITIONS AND METHODS OF USE

(71) Applicant: Bacainn Biotherapeutics, Ltd., George Town, Grand Cayman, (KY)

(72) Inventor: Chris Murphy, Upton, MA (US)

(73) Assignee: Bacainn Biotherapeutics, Ltd., George Town, Grand Cayman, (KY)

(21) Appl. No.: 18/556,019

(22) PCT Filed: Apr. 18, 2022
(86) PCT No.: PCT/US2022/025189

§ 371 Date: Oct. 18, 2023

Related U.S. Application Data
(60) Provisional application No. 63/177,238, filed on Apr. 20, 2021.

(51) Int. Cl. A61K 38/13; A61K 45/06; A61P 3/04; A61P 3/10

(52) U.S. Cl. CPC A61K 38/13; A61K 45/06; A61P 3/04; A61P 3/10

(57) ABSTRACT

Disclosed herein are cyclosporine compounds and methods for use in the treatment, prevention, or amelioration of glucose intolerance, wherein the compounds inhibit the activity of N-formyl peptide receptor 1 (FPR1).

### CROSS-REFERENCE TO RELATED APPLICATION

The present application is a 371(c) National Stage entry of PCT/US2022/025189, filed Apr. 18, 2022, which claims priority to U.S. Provisional Patent Application No. 63/177,238, filed on Apr. 20, 2021, the contents of which are hereby incorporated by reference in their entirety.

### TECHNICAL FIELD

The technology of the present disclosure relates to methods, compounds, and compositions for treating or preventing disease associated with glucose intolerance.

### BACKGROUND

Inflammation, and in particular chronic inflammation or chronic inflammatory disease (CID), is globally highly prevalent and is viewed as one of the major causes for the development of many different diseases. Inflammatory stress is closely related to metabolic disease, insulin resistance, and glucose intolerance. Although the precise cellular mechanism linking obesity and diabetes is largely unknown, many obese individuals develop glucose intolerance and chronic inflammation, which are associated with diseases and syndromes including not only obesity, but also diabetes and its complications, insulin resistance, metabolic syndrome, cardiovascular disease, cancer, osteoporosis, rheumatoid arthritis, inflammatory bowel disease, lung diseases such as asthma and chronic obstructive pulmonary disease (COPD), and CNS related diseases such as depression, Parkinson's disease, and Alzheimer's disease. N-formyl peptides, such as fMet-Leu-Phe (fMLP or fMLF), which act through binding to N-formyl peptide receptors (FPRs) such as FPR1 leading to inflammation, have been shown to be altered in conditions associated with glucose intolerance, such as obesity, and can directly impair glucose homeostasis. Hence, inhibition of FPRs, such as FPR1, is an avenue for the treatment or prevention of diseases and conditions associated with glucose intolerance.

### SUMMARY

In one aspect, the present disclosure relates to a method for treating glucose intolerance in a mammalian subject in need thereof, comprising administering to the subject a therapeutically effective amount of a compound of Formula I:

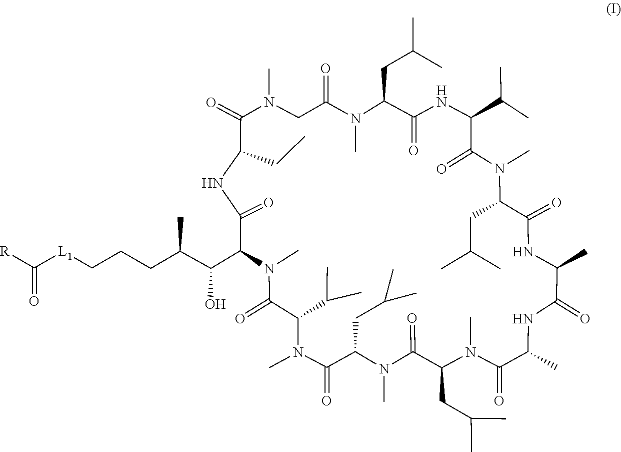

a stereoisomer thereof, or a pharmaceutically acceptable salt of any of the foregoing; wherein L₁ is a C₀ to C₄ alkyl group optionally substituted with one or more F; R is selected from the group consisting of —OH and a PEG, wherein the PEG optionally comprises a linker group, L₂ as defined herein. In some embodiments, R is selected from —OH or —NH(CH₂)₂₋₆(CH₂CH₂O)₄₂₋₄₆—O(CH₂)₀₋₅CH₃. In some embodiments, R is selected from the group consisting of CH₃O—(CH₂ CH₂₀)44—CH₂CH₂CH₂NH— (BT051), CH₃O—(CH₂CH₂O)₄₄—CH₂CH₂NH— (BT090), and HO— (BT070). In some embodiments, L₁ is an ethylene group or a propylene group. In some embodiments, R is a PEG having from 40 to 50 ethylene oxide units. In some embodiments, L₂ is a substituted or unsubstituted heteroalkylene group.

In some embodiments, the compound of Formula I has the Formula IA, or a pharmaceutically acceptable salt thereof:

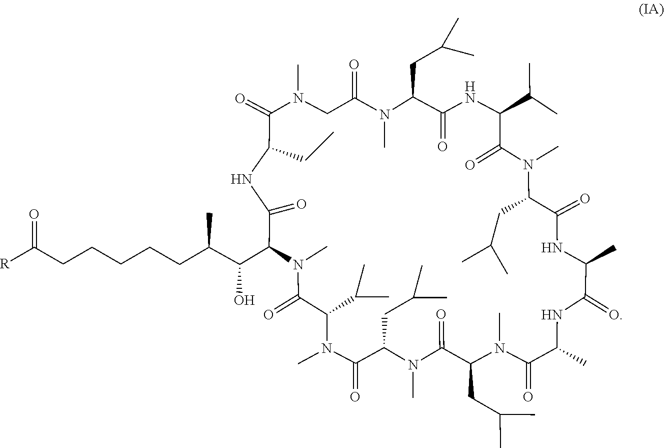

In some embodiments, the compound is formulated as a pharmaceutical composition comprising the compound and a pharmaceutically acceptable carrier.

In some embodiments, the glucose intolerance is obesity-induced.

In some embodiments, the glucose intolerance is associated with one or more diseases selected from the group consisting of type 2 diabetes mellitus, type 1 diabetes mellitus, gestational diabetes mellitus, neonatal diabetes, maturity-onset diabetes of the young, drug-induced diabetes, chemical-induced diabetes, hyperglycemia, impaired glucose tolerance (IGT), impaired fasting glucose (IFT), obesity, insulin resistance, metabolic syndrome, asthma, chronic obstructive pulmonary disease (COPD), Alzheimer's disease, cancers, Klinefelter Syndrome, Turner Syndrome, Wolfram Syndrome, Cystic Fibrosis, Friedreich's ataxia, multiple sclerosis, down syndrome, muscular dystrophy, polycystic ovary syndrome (PCOS), uremia, cirrhosis, chronic renal failure, and HAART-treated HIV infection.

In some embodiments, the disease is obesity, and the obesity is diet-induced obesity.

In some embodiments, treatment comprises one or more of improved glucose tolerance, improved insulin sensitivity, and increased plasma glucagon-like peptide 1 (GLP-1) levels as compared to untreated control subjects.

In some embodiments, the method further comprises administering to the subject a therapeutically effective amount of one or more second compound that inhibits one or more of multidrug resistance protein 2 (MRP2) and HXA3 synthase, wherein the therapeutic amount of the second compound reduces migration of neutrophils into the target tissue.

In some embodiments, the method further comprises administering to the subject a therapeutically effective amount of one or more compounds that increases multidrug resistance protein 1 (MRP1), wherein the therapeutic amount of the third compound reduces migration of neutrophils into the target tissue.

In some embodiments, the method further comprises administering one or more compounds that inhibits one or more of multidrug resistance protein 2 (MRP2) and hepoxilin A3 (HXA3) synthase, wherein the therapeutic amount of the second compound reduces migration of neutrophils into the target tissue.

In some embodiments, the method further comprises administering to the subject a therapeutically effective amount of one or more compounds that increases one or more N-acylethanolamines (NAEs), wherein the therapeutic amount of the third compound reduces migration of neutrophils into the target tissue.

In some embodiments, the method further comprises administering one or more antibiotic and/or anti-inflammatory agents selected from the group consisting of: Dalbavancin, Oritavancin, Cubicin, Tedizolid, Ceftobiprole, Ceftobiprole, Ceftolozane-tazobactam, mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, steroidal agents such as corticosteroids such as hydrocortisone, hydroxyltriamcinolone alphamethyl dexamethasone, dexamethasonephosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, non-steroidal agents such as COX inhibitors, LOX inhibitors, p38 kinase inhibitors, immunosuppresant agents such as cyclosporin, and cytokine synthesis inhibitors, tetracycline, minocycline, and doxycycline, or any combination thereof.

In some embodiments, the method further comprises administering one or more agents selected from the group consisting of: metformin; incretin mimetics; DPP-4-resistant GLP-1 receptor agonists, such as exenatide and liraglutide, DPP-4 inhibitors (sitagliptin, vildagliptin); amylin; pramlintide acetate; oral hypoglycemic drugs; orlistat; insulin; insulin degludec; insulin detemir; human insulin; biphasic human insulin; insulin aspart; biphasic insulin aspart; glucagon-like peptide-1; semaglutide; and repaglinide; or any combination thereof.

### BRIEF DESCRIPTION OF THE DRAWINGS

FIG. 1 is a chart showing the dose-response of BT051 and BT070 on the inhibition of neutrophil migration.

FIGS. 2A, 2B, and 2C. FIG. 2A is a chart showing the stability of BT090 in simulated intestinal fluid (SIF). FIG. 2B is a chart showing the stability of BT090 in simulated gastric fluid (SGF). FIG. 2C is a chart showing the stability of BT090 in feces.

FIGS. 3A and 3B are charts showing the inhibition of formyl peptide receptor 1 (FPR1) by BT051 (FIG. 3A) and BT051 and BT070 (FIG. 3B) as measured by the inhibition of fMLP-mediated neutrophil transmigration/activation.

FIGS. 4A and 4B are charts showing the body weight and weight gain of diet-induced obese (DIO) mice during the acclimation period.

FIGS. 5A and 5B are charts showing the weight gain of BT051-treated mice and vehicle-treated control mice during the treatment period. Non-significant with a two-way ANOVA and Bonferroni's post-test.

FIGS. 6A-6C are charts showing the group average and individual changes to fasting blood glucose levels (mg/dL) in mice before and after treatment with BT051 or vehicle. FIGS. 6D-6F are charts showing the group average and individual changes to plasma insulin levels (μU/mL) in DIO mice before and after treatment with BT051 or vehicle. FIGS. 6G-6I are charts showing the group average and individual changes to HOMA-IR index values in DIO mice before and after treatment with BT051 or vehicle. # p<0.05 with a Student's t-test.

FIGS. 7A-7G are charts showing the oral glucose tolerance testing results in DIO mice treated with either BT051 or vehicle. FIG. 7A is a chart of the blood glucose curve (mg/dL) over the course of the experimental period, with FIG. 7B showing the area under the curve (AUC) (mg/dL.min) over the entire time course, and FIG. 7C showing the AUC (mg/dL.min) from 0-60 minutes from oral glucose loading. FIGS. 7D-7F are charts showing the group average and individual changes to plasma insulin levels (μU/mL) at −30 minutes from glucose loading and 15 minutes from glucose loading. FIG. 7G is a chart of the T15/-30 plasma insulin ratio. Non-significant with a two-way ANOVA and Bonferroni's post-test (FIG. 7A); ## p<0.01 and #### p<0.0001 with a Mann-Whitney test (FIGS. 7D-F).

### DETAILED DESCRIPTION

### I. Definitions

The following terms are used herein, the definitions of which are provided for guidance.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. It will be understood by those of skill in the art that substituted groups of the present technology are chemically stable groups that allow isolation of the compounds in which they appear. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; sulfonamides; amines; N-oxides; azides; amides; ureas; amidines; guanidines; nitro groups; nitriles (i.e., CN); and the like.

Alkyl groups include straight chain and branched chain alkyl groups having (unless indicated otherwise) from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like. In some embodiments the alkyl group is substituted with 1, 2, or 3 substituents.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Heteroalkyl groups and heteroalkenyl groups are, respectively, alkyl groups (as defined herein) and alkenyl groups (as defined herein) that include from 1 to 6 heteroatoms selected from N, O and S. It will be understood that each heteroatom present is bonded to at least one carbon atom within the heteroalkyl or heteroalkenyl group. In some embodiments the heteroaklyl or heteteroalkenyl groups include 1, 2, or 3 heteroatoms. Heteroalkyl and heteroalkenyl groups may be substituted or unsubstituted. Examples of heteroalkyl groups include but are not limited to CH₃CH₂OCH₂, CH₃NHCH₂, CH₃CH₂N(CH₃)CH₂, CH₃CH₂SCH₂, CH₃CH₂OCH₂CH₂OCH₂CH₂. Examples of heteroalkenyl groups include but are not limited to CH₂ CHOCH₂, CH₂=CHN(CH₃)CH₂, and CH₂═CHSCH₂.

Representative substituted heteroalkyl or heteroalkeneyl groups may be substituted one or more times with substituents such as those listed above (e.g., 1, 2 or 3 times), and include without limitation haloheteroalkyl (e.g., trifluoromethyloxyethyl), carboxyalkylaminoalkyl, methyl acrylate and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1 ]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4- 2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent cycloalkyl groups are cycloalkylene groups, divalent heteroalkyl groups are heteroalkylene groups, divalent alkenyl groups are alkenylene groups, and so forth. Substituted groups having a single point of attachment to the compound of the present technology are not referred to with the “ene” designation. Thus, e.g., chloroethyl is not referred to herein as chloroethylene.

The term “administering” a molecule to a subject means delivering the molecule to the subject. “Administering” includes prophylactic administration of the composition (i.e., before the disease and/or one or more symptoms of the disease are detectable) and/or therapeutic administration of the composition (i.e., after the disease and/or one or more symptoms of the disease are detectable). The methods of the present technology include administering one or more compounds. If more than one compound is to be administered, the compounds may be administered together at substantially the same time, and/or administered at different times in any order. Also, the compounds of the present technology may be administered before, concomitantly with, and/or after administration of another type of drug or therapeutic procedure (e.g., surgery).

The terms “alter” and “modify” when in reference to the level of any molecule (e.g., fMLP, multidrug resistance protein 2 (MRP2), multidrug resistance protein 1 (MRP1), hepoxilin A3 (HXA₃) synthase, N-acyl ethanolamine (NAE), amino acid sequence, nucleic acid sequence, antibody, etc.), cell, and/or phenomenon (e.g., level of activity of fMLP and/or multidrug resistance protein 2 (MRP2) and/or of multidrug resistance protein 1 (MRP1) and/or of hepoxilin A3 (HXA₃) synthase and/or N-acyl ethanolamine (NAE), level of expression of a gene, disease symptom, level of binding of two molecules such as binding of a hormone ligand to its hormone receptor, specificity of binding of two molecules, affinity of binding of two molecules, disease symptom, specificity to disease, sensitivity to disease, affinity of binding, enzyme activity, etc.) in a first sample (or in a first subject) relative to a second sample (or relative to a second subject), refer to an increase and/or decrease.

“Cannabinoid receptor type 2” (“CB2”) is a G protein-coupled receptor from the cannabinoid receptor family that in humans is encoded by the CNR2 gene. The principal endogenous ligand for the CB2 receptor is 2-arachidonoylglycerol (2-AG).

Use of the terms “comprising”, “including” or similar terms to describe or define an embodiment of a compound, composition or method having one or more elements shall be understood to also disclose embodiments “consisting” or “consisting essentially” of the elements and vice versa. In other words, disclosure of embodiments open to elements beyond those listed (“comprising”), also are to be understood to disclose embodiments which are closed to additional elements (“consisting”) or which may only include additional elements that do not materially affect the characteristics of the embodiment (“consisting essentially”). Likewise, embodiments consisting or consisting essentially of the listed elements shall be understood to disclose embodiments comprising those elements.

The term “conjugating,” and grammatical equivalents, when made in reference to conjugating a molecule of interest and a polymer means covalently linking the molecule of interest to the polymer. Linkage may be direct. Alternatively, linkage may be indirect via a linking group or moiety. Methods for conjugation to polymers are known in the art, including methods for conjugation to a polypeptide to produce a fusion protein (Pasut, Polymers 6:160-178 (2014); Medscape, Nanomedicine 5(6):915-935 (2010)). In some embodiments, the conjugate comprises cyclosporine A conjugated to a PEG polymer. Precursors to such cyclosporine A conjugates, include cyclosporine A modified, e.g., with a linking group, but without the PEG polymer. The linking group links the polymer to the cyclosporine A.

As used herein, the terms “effective amount” or “therapeutically effective amount,” or “pharmaceutically effective amount” refer to a quantity sufficient to achieve a desired therapeutic and/or prophylactic effect, e.g., an amount which results in the full or partial amelioration of glucose intolerance or disease or disorders or symptoms associated with glucose intolerance in a subject in need thereof. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds. In some embodiments, multiple doses are administered. Additionally or alternatively, in some embodiments, multiple therapeutic compositions or compounds are administered. In the methods described herein, the therapeutic compounds may be administered to a subject having one or more signs or symptoms of a disease or disorder associated with glucose intolerance.

“Endocannabinoids” (“ECs”) are compounds that bind to the cannabinoid receptors, CB1 and CB2, as well as more recently described atypical receptors GPR55 and GPR119. The two main classes of eicosanoid-type ECs are “N-acylethanolamines” (“NAEs”) and monoacylglycerols (MAGs), which are metabolized by fatty acid amide hydrolase (FAAH) and monoacyl glycerol lipase (MAGL), respectively. “N-acylethanolamine” is an endocannabinod and is a type of fatty acid amide formed when one of several types of acyl group is linked to the nitrogen atom of ethanolamine. N-acylethanolamines are metabolized by fatty acid amide hydrolase (FAAH). Exemplary N-acylethanolamine endocannabinoids include ethanolamine, anandamide (AEA) (N-arachidonoylethanolamine), which is the amide of arachidonic acid (20:4 ω-6) oleoyl ethanolamide (OEA), and alpha-linolenoyl ethanolamide (α-LEA).

“Fatty acid amide hydrolase,” “FAAH,” and “EC 3.5.1.99” interchangeably refer to a member of the serine hydrolase family of enzymes. It was first shown to break down anandamide. In humans, it is encoded by the gene FAAH.

“Hepoxilin A3 synthase,” “HXA₃ synthase,” “ALOX12,” “12-lipoxygenase,” “arachidonate 12-lipoxygenase,” “12S-Lipoxygenase,” “12-LOX,” and “12S-LOX” interchangeably refer to a lipoxygenase-type enzyme (i.e., an enzyme that catalyzes the dioxygenation of polyunsaturated fatty acids in lipids containing a cis, cis-1,4- pentadiene structure) that in humans is encoded by the ALOX12 gene, which is located along with other lipoyxgenases on chromosome 17p13.3.

The term “increase” when in reference to a compound means increase the level and/or activity of the compound. The terms “increase,” “elevate,” “raise,” and grammatical equivalents (including “higher,” “greater,” etc.) when in reference to the level of any molecule, cell, and/or phenomenon, level of expression of a gene, disease symptom, level of binding of two molecules such as binding of a hormone ligand to its hormone receptor, specificity of binding of two molecules, affinity of binding of two molecules, disease symptom, specificity to disease, sensitivity to disease, affinity of binding, enzyme activity, etc.) in a first sample (or in a first subject) relative to a second sample (or relative to a second subject), mean that the quantity of the molecule, cell and/or phenomenon in the first sample (or in the first subject) is higher than in the second sample (or in the second subject) by any amount that is statistically significant using any art-accepted statistical method of analysis. In one embodiment, the quantity of the molecule, cell and/or phenomenon in the first sample (or in the first subject) is at least 10% greater than, at least 25% greater than, at least 50% greater than, at least 75% greater than, and/or at least 90% greater than the quantity of the same molecule, cell and/or phenomenon in the second sample (or in the second subject). This includes, without limitation, a quantity of molecule, cell, and/or phenomenon in the first sample (or in the first subject) that is at least 10% greater than, at least 15% greater than, at least 20% greater than, at least 25% greater than, at least 30% greater than, at least 35% greater than, at least 40% greater than, at least 45% greater than, at least 50% greater than, at least 55% greater than, at least 60% greater than, at least 65% greater than, at least 70% greater than, at least 75% greater than, at least 80% greater than, at least 85% greater than, at least 90% greater than, and/or at least 95% greater than the quantity of the same molecule, cell and/or phenomenon in the second sample (or in the second subject). In one embodiment, the first sample (or the first subject) is exemplified by, but not limited to, a sample (or subject) that has been manipulated using the compositions and/or methods of the present technology. In a further embodiment, the second sample (or the second subject) is exemplified by, but not limited to, a sample (or subject) that has not been manipulated using the compositions and/or methods of the present technology. In an alternative embodiment, the second sample (or the second subject) is exemplified by, but not limited to, a sample (or subject) that has been manipulated, using the compositions and/or methods of the present technology, at a different dosage and/or for a different duration and/or via a different route of administration compared to the first subject. In one embodiment, the first and second samples (or subjects) may be the same, such as where the effect of different regimens (e.g., of dosages, duration, route of administration, etc.) of the compositions and/or methods of the present technology is sought to be determined on one sample (or subject). In another embodiment, the first and second samples (or subjects) may be different, such as when comparing the effect of the compositions and/or methods of the present technology on one sample (subject), for example a patient participating in a clinical trial and another individual in a hospital.

The term “inhibit” when used in reference to a compoundmeans inhibit the activity and/or level of the compound. The terms “inhibit,” “reduce,” “diminish,” “suppress,” “decrease,” and grammatical equivalents (including “lower,” “smaller,” etc.) when in reference to the level of any molecule, cell, and/or phenomenon, level of expression of a gene, disease symptom, level of binding of two molecules such as binding of a hormone ligand to its hormone receptor, specificity of binding of two molecules, affinity of binding of two molecules, disease symptom, specificity to disease, sensitivity to disease, affinity of binding, enzyme activity, etc.) in a first sample (or in a first subject) relative to a second sample (or relative to a second subject), mean that the quantity of molecule, cell, and/or phenomenon in the first sample (or in the first subject) is lower than in the second sample (or in the second subject) by any amount that is statistically significant using any art-accepted statistical method of analysis. In one embodiment, the quantity of molecule, cell and/or phenomenon in the first sample (or in the first subject) is at least 10% lower than, at least 25% lower than, at least 50% lower than, at least 75% lower than, and/or at least 90% lower than the quantity of the same molecule, cell and/or phenomenon in the second sample (or in the second subject). In another embodiment, the quantity of molecule, cell, and/or phenomenon in the first sample (or in the first subject) is lower by any numerical percentage from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100% lower than the quantity of the same molecule, cell and/or phenomenon in the second sample (or in the second subject). In one embodiment, the first sample (or the first subject) is exemplified by, but not limited to, a sample (or subject) that has been manipulated using the compositions and/or methods of the present technology. In a further embodiment, the second sample (or the second subject) is exemplified by, but not limited to, a sample (or subject) that has not been manipulated using the compositions and/or methods of the present technology. In an alternative embodiment, the second sample (or the second subject) is exemplified by, but not limited to, a sample (or subject) that has been manipulated, using the compositions and/or methods of the present technology, at a different dosage and/or for a different duration and/or via a different route of administration compared to the first subject. In one embodiment, the first and second samples (or subjects) may be the same, such as where the effect of different regimens (e.g., of dosages, duration, route of administration, etc.) of the compositions and/or methods of the present technology is sought to be determined on one sample (or subject). In another embodiment, the first and second samples (or subjects) may be different, such as when comparing the effect of the compositions and/or methods of the present technology on one sample (subject), for example a patient participating in a clinical trial and another individual in a hospital.

“Multidrug resistance-associated protein 2,” “multidrug resistance protein 2” (“MRP2”), “canalicular multispecific organic anion transporter 1” (“cMOAT”), “ATP-binding cassette sub-family C member 2” (“ABCC₂”) are interchangeably used to refer to protein that in humans is encoded by the AB(('2 gene.

“Multidrug resistance protein 1,” “MRP1” and “ABCC₁” are interchangeably used to refer to a uni-directional efflux transporter protein with a wide substrate specificity including important therapeutics. Some of the main roles of this transporter are: (i) efflux of xenobiotic and endogenous metabolites; (ii) transport of inflammatory mediators (e.g., LTC₄); and (iii) defense against oxidative stress. The 190-kDa MRP1 has a core structure consisting of two trans-membrane domains (TMD), each followed by a nucleotide binding domain (NBD). In common with MRP2, 3, 6, and 7, MRP1 contains a third TMD (TMDO) with five predicted trans membrane segments and an extra cytosolic NH₂ terminus connected to the core structure by a linker region (LO) (Rosenberg et al., J. Biol. Chem. 276(19): 13076-16082 (2001)). The TMDO appears to be important for MRPI trafficking to the plasma membrane (Bakos et al., J. Cell Sci. 113(Pt 24):4451-4461 (2000)), and the precise roles, mechanisms, and dependencies of TMDO and LO are the subject of significant research (Westlake et al. Mol. Biol. Cell 16(5):2483-2492 (2005)). MRP1 has broad substrate specificity, transporting hydrophobic and anionic molecules, glucuronide and glutathione conjugates, as well as endogenous glutathione. Although many MRP1 substrates are conjugated to glutathione, co-transport of free glutathione is often observed, and appears to stimulate transport of e.g., vincristine and daunorubicin (Hooijberga et al., FEBS Letters 469:47-51(2000)). Glutathione itself is a low affinity substrate of MRPI (Km=1-5 mM). Multiple allosterically cooperative, non-overlapping substrate-binding sites are postulated, which may explain why various substrates both cross-inhibit and cross-stimulate (Bakos et al., Pflugers Arch-Eur J Physiol 453:621-641(2007)). The inflammatory cytokine LTC₄ and its main metabolite LTD4 are some of the highest affinity MRP1 substrates, suggesting a key role for MRP1 in cytokine release from LTC₄ producing cells. In fact, intracellular LTC₄ accumulation was observed in mrp1 (−/−) mice (Robbiani et al., Cell 103:757-768 (2000)). Additionally, although viable, healthy, and fertile with normal phenotype, knockout mrp1 (−/−) mice were hypersensitive to cytotoxic drugs (Wijnholds et al., Nat. Med. 3:1275-1279 (1997)). MRP1 is exemplified by the human protein sequence NCBI Reference Sequence: NP_004987.2 encoded by the DNA sequence NCBI Reference Sequence: NG_028268.1. There are at least 15 naturally occurring mutations identified in MRP1, and many of them have been found to affect its in vitro transport activity. Polymorphisms and mutagenesis studies have been reviewed in He et al., Curr. Med. Chem. 18:439-481 (2011). Although many MRP1 SNPs are known, their incidence in populations is reported to be relatively low. In mainland Chinese populations the MRPI polymorphism allelic frequencies of Cys43Ser (128G>C), Thr73Ile (218C>T), Arg723Gln (2168G>A) and Arg1058Gln (3173G>A) were 0.5%, 1.4%, 5.8% and 0.5%, respectively (Ji-Ye Yin et al., Pharmacogenet. Genomics 19(3):206-216 (2009)).

“P-glycoprotein” (“P-gp”) is an efflux membrane transporter, and is responsible for limiting cellular uptake and the distribution of xenobiotics and toxic substances.

Pharmaceutically acceptable salts of compounds described herein are within the scope of the present technology and include acid or base addition salts which retain the desired pharmacological activity and are not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g. dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

“Polymer” is a substance that has a molecular structure consisting chiefly or entirely of a large number of similar units bonded together. Polymers may occur naturally (e.g., cellulose, polypeptides, nucleotides sequences, etc.) or are artificial (e.g., plastics, resins, etc.). Polymers may be used as carriers of drugs to which they are conjugated, and may enhance the solubility of the conjugated drug, improve its pharmacokinetic profile, protect the drug against degradation, release the drug under certain conditions, such as change in pH or in the presence of enzymes, such as esterases, lipases or proteases. In addition, a targeting moiety or a solubilizer may also be introduced into the conjugate to boost its therapeutic index (Medscape, Nanomedicine 5(6):915-935(2010)). Polymers may also be utilized to restrict the distribution of the drug conjugated to it by, for example, preventing the conjugated drug from crossing into specific body compartments (e.g., from the gastrointestinal lumen to the underlying tissue). Polymers may be natural polymers and/or synthetic linear polymers, and include polyethylene glycol (PEG), dextran, periodate-oxidized dextran, polysialic acids (PSAs), hyaluronic acid (HA), dextrin, hydroxyethyl-starch (HES), poly(2-ethyl 2-oxazoline) (PEOZ), polyglutamic acid (PGA), polylactic acid (PLA), polylactic-co-glycolic (PLGA), poly(D,L-lactide-co- glycolide) (PLA/PLGA), poly(hydroxyalkylmethaacrylamide), polyglycerol, 25 polyamidoamine (PAMAM), polyethylenimine (PEI), and polypeptides.

“SipA” and “Salmonella T3SS effector protein” are used interchangeably to refer to a protein produced by Salmonella, as exemplified by the amino acid sequence of Salmonella enterica subsp. enterica serovar Typhimurium str. SL1344 (GenBank: AAA86618.1) encoded by the DNA sequence (Locus taq) SL1344_2861 of the Salmonella enterica subsp. enterica serovar Typhimurium str. SL1344, complete genome sequence (NCBI Reference Sequence: NC_016810.1). The SipA sequence is provided by WO 2015/089268.

“Target tissue” that may suffer from inflammation includes, without limitation, epithelial tissue, mucosal tissue, etc. Exemplary epithelial tissue and/or mucosal tissue include gastrointestinal, lung (e.g., bronchial tissue), liver, stomach, colon, brain, gallbladder, renal, female genital tract, ocular, urinary tract, etc., resulting in “inflammatory diseases” such as glucose intolerance and/or diseases or conditions associated with glucose intolerance.

“Treating,” “treat,” “treated,” or “treatment” as used herein covers the treatment of a disease or disorder described herein (e.g., glucose intolerance and/or diseases or disorders associated with glucose intolerance, or inflammation), in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. Symptoms may be assessed by methods known in the art, for example, biopsy and histology, and blood tests to determine relevant enzyme levels, metabolites or circulating antigen or antibody (or other biomarkers), quality of life questionnaires, patient-reported symptom scores, and imaging tests.

As used herein, “prevention” or “preventing” of a disorder or condition described herein (e.g., glucose intolerance and/or diseases or disorders associated with glucose intolerance, or inflammation) refers to a compound that, in a statistical sample, reduces the occurrence of the disorder or condition in the treated sample relative to a control sample, or delays the onset of one or more symptoms of the disorder or condition relative to the control sample.

It is also to be appreciated that the various modes of treatment or prevention of medical diseases and conditions as described are intended to mean “substantial,” which includes total but also less than total treatment or prevention, and wherein some biologically or medically relevant result is achieved.

As used herein, the terms “subject,” “individual,” or “patient” can be an individual organism, a vertebrate, a mammal, or a human. “Mammal” includes a human, non-human primate, murine (e.g., mouse, rat, guinea pig, hamster), ovine, bovine, ruminant, lagomorph, porcine, caprine, equine, canine, feline, ave, etc. In some embodiments, the mammal is murine. In some embodiments, the mammal is human.

A subject “in need” of treatment according to the methods and/or compositions of the present technology includes a subject that is “suffering” from glucose intolerance and/or diseases or disorders associated with glucose intolerance, or inflammation (i.e., a subject that is experiencing and/or exhibiting one or more clinical and/or subclinical symptoms of glucose intolerance and/or diseases or disorders associated with glucose intolerance, or inflammation), and a subject “at risk” of glucose intolerance and/or diseases or disorders associated with glucose intolerance, or inflammation . A subject “in need” of treatment includes animal models of glucose intolerance and/or diseases or disorders associated with glucose intolerance, or inflammation. Subject “at risk” of glucose intolerance and/or diseases or disorders associated with glucose intolerance, or inflammation refers to a subject that is not currently exhibiting glucose intolerance and/or diseases or disorders associated with glucose intolerance, or inflammation symptoms and is predisposed to expressing one or more symptoms of the disease or disorder. This predisposition may be based on family history, genetic factors, environmental factors such as exposure to detrimental compounds present in the environment, etc. It is not intended that the present technology be limited to any particular signs or symptoms. Thus, it is intended that the present technology encompass subjects that are experiencing any range of disease, from sub-clinical symptoms to full-blown glucose intolerance and/or diseases or disorders associated with glucose intolerance, or inflammatory disease, wherein the subject exhibits at least one of the indicia (e.g., signs and symptoms) associated with the glucose intolerance and/or diseases or disorders associated with glucose intolerance, or inflammatory disease.

“Substantially the same,” “without substantially altering,” “substantially unaltered,” and grammatical equivalents, when in reference to the level of any molecule, cell, and/or phenomenon means that the quantity of molecule, cell, and/or phenomenon in the first sample (or in the first subject) is neither increased nor decreased by a statistically significant amount relative to the second sample (or in a second subject). Thus, in one embodiment, the quantity of molecule, cell, and/or phenomenon in the first sample (or in the first subject) is from 90% to 100% (including, for example, from 91% to 100%, from 92% to 100% , from 93% to 100%, from 94% to 100%, from 95% to 100%, from 96% to 100%, from 97% to 100%, from 98% to 100%, and/or from 99% to 100%) of the quantity in the second sample (or in the second subject).

As used herein, “weight percent” of a component, unless specifically stated to the contrary, is based on the total weight of the formulation or composition in which the component is included.

### II. General

In one aspect, the present technology provides methods, compounds, and compositions for inhibiting formyl peptide receptor 1 (FPR1) and for treating disease associated with FPR1 activation. In some embodiments, disease associated with FPR1 activation comprises glucose intolerance and/or diseases or disorders associated with glucose intolerance.

Heterogeneity occurs in most glucose intolerance disorders, including diabetes mellitus syndromes.

Type 1 diabetes mellitus. Type 1 diabetes is characterized by absolute insulin deficiency. In type 1A, a cellular-mediated autoimmune destruction of beta cells of the pancreas occurs. The disease process is initiated by an environmental factor—that is, an infectious or noninfectious agent in genetically susceptible individuals. Some genes in the histocompatibility leukocyte antigen (HLA) system are thought to be crucial. A stress-induced epinephrine release, which inhibits insulin release (with resultant hyperglycemia), sometimes occurs and may be followed by a transient asymptomatic period known as “the honeymoon.” Lasting weeks to months, the honeymoon precedes the onset of overt, permanent diabetes.

Amylin, a beta-cell hormone that is normally cosecreted with insulin in response to meals, is also completely deficient in persons with type 1 diabetes mellitus. Amylin exhibits several glucoregulatory effects that complement those of insulin in postprandial glucose regulation. Idiopathic forms of type 1 diabetes also occur, without evidence of autoimmunity or HLA association; this subset is termed type 1B diabetes.

The underlying pathophysiology of beta cell demise or dysfunction is currently more understood in type 1 diabetes than in type 2 diabetes. The rate of progression in type 1 diabetes is dependent on the age at first detection of antibody, number of antibodies, antibody specificity, and antibody titer. Three distinct stages of type 1 diabetes have been recognized. Both stages 1 and 2 are characterized by autoimmunity and a presymptomatic status; although there is still normoglycemia in stage 1, dysglycemia (impaired fasting glucose [IFG] and/or impaired glucose tolerance [IGT]) is present in stage 2. Stage 3 is characterized by new-onset symptomatic hyperglycemia.

Type 2 diabetes mellitus. In a state of health, normoglycemia is maintained by fine hormonal regulation of peripheral glucose uptake and hepatic production. Type 2 diabetes mellitus results from a defect in insulin secretion and an impairment of insulin action in hepatic and peripheral tissues, especially muscle tissue and adipocytes. A postreceptor defect is also present, causing resistance to the stimulatory effect of insulin on glucose use. As a result, a relative insulin deficiency develops, unlike the absolute deficiency found in patients with type 1 diabetes. The specific etiologic factors are not known, but genetic input is much stronger in type 2 diabetes than in the type 1 form.

Impaired glucose tolerance (IGT) is a transitional state from normoglycemia to frank diabetes, but patients with impaired glucose tolerance exhibit considerable heterogeneity. Type 2 diabetes, or glucose intolerance, is part of a dysmetabolic syndrome (syndrome X) that includes insulin resistance, hyperinsulinemia, obesity, hypertension, and dyslipidemia. Current knowledge suggests that the development of glucose intolerance or diabetes is initiated by insulin resistance and worsened by the compensatory hyperinsulinemia. Insulin resistance is not only predictive for type 2 diabetes and associated with myriad metabolic derangements in fasting conditions, but nondiabetic insulin-resistant individuals are subjected to a similar adverse postprandial metabolic setting and cardiometabolic risk as those with type 2 diabetes. In addition, the prevalence of hypertension rises with exacerbation of stages of impaired glucose metabolism; however, only in the early stages of impaired insulin metabolism do hyperglycemia and hyperinsulinemia appear to be significant contributors to the presence of hypertension.

The paths to beta-cell dysfunction or demise are less well defined in type 1 diabetes. The progression to type 2 diabetes is influenced by genetics and environmental or acquired factors, such as a sedentary lifestyle and dietary habits that promote obesity. Most patients with type 2 diabetes are obese, and obesity is associated with insulin resistance. Insulin resistance is not only predictive for type 2 diabetes and associated with myriad metabolic derangements in fasting conditions, but nondiabetic insulin-resistant individuals are subjected to a similar adverse postprandial metabolic setting and cardiometabolic risk as those with type 2 diabetes. Central adiposity is more important than increased generalized fat distribution. In patients with frank diabetes, glucose toxicity and lipotoxicity may further impair insulin secretion by the beta cells. Moreover, in obesity, inflammation, with increased accumulation and inflammatory polarization of immune cells, takes place in various tissues, including adipose tissue, skeletal muscle, liver, gut, pancreatic islet, and brain, and may contribute to obesity-linked metabolic dysfunctions, leading to insulin resistance and type 2 diabetes.

Gestational diabetes mellitus (GDM). GDM was previously described as any degree of glucose intolerance in which onset or first recognition occurs during pregnancy. The definition was limited by imprecision. Women diagnosed with diabetes in the first trimester are now classified as having type diabetes. GDM is diabetes diagnosed in the second or third trimester of pregnancy that is not clearly overt diabetes. Insulin requirements are increased during pregnancy because of the presence of insulin antagonists, such as human placental lactogen or chorionic somatomammotropin, and cortisol; these promote lipolysis and decrease glucose use.

Another factor in increased insulin requirements during pregnancy is the production of insulinase by the placenta. Various genetic defects of the beta cell, insulin action, diseases of the exocrine pancreas, endocrinopathies, drugs, chemical agents, infections, immune disorders, and genetic syndromes can cause variable degrees of glucose intolerance, including diabetes.

Other specific types of diabetes mellitus. There are specific types of diabetes due to other causes, which include monogenic diabetes syndromes, diseases of the exocrine pancreas, and drug- or chemical induced diabetes. Various genetic defects of the beta cell, insulin action, diseases of the exocrine pancreas, endocrinopathies, drugs, chemical agents, infections, immune disorders, and genetic syndromes can cause variable degrees of glucose intolerance, including diabetes.

Varying forms of glucose intolerance. Glucose intolerance may be present in many patients with cirrhosis due to decreased hepatic glucose uptake and glycogen synthesis. Other underlying mechanisms include hepatic and peripheral resistance to insulin and serum hormonal abnormalities. Abnormal glucose homeostasis may also occur in uremia, as a result of increased peripheral resistance to the action of insulin.

The gastrointestinal tract plays a significant role in glucose tolerance. With food ingestion, incretin hormones glucagonlike peptide-1 (GLP-1) and glucose-dependent insulinotropic polypeptide (GIP) are synthesized and secreted by specialized gut cells. Oral glucose administration results in a higher insulin secretory response than does intravenous glucose administration; this difference is due in part to incretin hormones.

The significance of incretin hormones has been noted as a result of efforts to develop agents that may improve glycemic control in patients with type 2 diabetes through new mechanisms. These strategies include inhibition of dipeptidyl peptidase IV (DPP-4), the major enzyme responsible for degrading incretin hormones in vivo, and the use of GLP-1 agonists. Incretin hormones also significantly affect the differentiation, mitogenesis, and survival of beta cells.

Pathologic defects observed in type 2 diabetes mellitus and sometimes in impaired glucose tolerance include postprandial hyperglucagonemia, dysregulation of gastric emptying, and loss of incretin effect.

Postprandial hyperglycemia in diabetes and impaired glucose tolerance (IGT) is related to a lower rate of glucose disposal, whereas insulin secretion and action, as well as postprandial turnover, are essentially normal in individuals with isolated IGT.

In addition to the diseases and disorders described above, glucose intolerance is associated with many additional human conditions such as insulin resistance and metabolic syndrome. Some of conditions are associated with genetic disorders, including Klinefelter Syndrome, Turner Syndrome, Wolfram syndrome, cystic fibrosis, and Friedrich's Ataxia. Glucose intolerance is also associated with multiple sclerosis, Down Syndrome, and muscular dystrophy. Other human conditions associated with glucose intolerance include polycystic ovary syndrome (PCOS), uremia, cirrhosis, chronic renal failure, and HIV infection when treated with HAART.

Standard methods and workups for diagnosing glucose intolerance are known in the art. As an illustrative, non-limiting example, the World Heath Organization uses the following diagnostic criteria for diabetes and impaired glucose intolerance: Diabetes: FPG≥126 mg/dL (7.0 mmol/L) or OGTT 2-hour PG ≥200 mg/dL (11.1 mmol/L); Impaired fasting glucose (IFG): FPG 110 mg/dL to 125 mg/dL (6.1 to 6.9mmol/L) and OGTT 2-hour PG <140 mg/dL (<7.8mmol/L); Impaired glucose tolerance (IGT): FPG <126 mg/dl (<7.0 mmol/L) and OGTT 2-hour PG 140mg/dl to 200mg/dl (≥7.8 and <11.1mmol/L).

In one aspect, the present technology provides methods, compounds, and compositions for inhibiting formyl peptide receptor 1 (FPR1) and for treating disease associated with FPR1 activation.

In one aspect, the present technology provides methods, compounds, and compositions for treating neutrophil-mediated inflammation and disease associated with neutrophil-mediated inflammation. In particular, the present technology provides a method for treating neutrophil-mediated inflammation in a target tissue of a mammalian subject in need thereof, comprising administering to the subject a therapeutically effective amount of one or more first compound that increases the level and/or activity of multidrug resistance protein 1 (MRP1), wherein the therapeutic amount of the first compound reduces migration of neutrophils into the target tissue, and/or administering a therapeutically effective amount of one or more second compound that inhibits one or more of multidrug resistance protein 2 (MRP2), and hepoxilin A3 (HXA₃) synthase, wherein the therapeutic amount of the second compound reduces migration of neutrophils into the target tissue, and/or administering a therapeutically effective amount of one or more third compound that increases one or more N-acylethanolamines (NAEs), wherein the therapeutic amount of the third compound reduces migration of neutrophils into the target tissue.

In one aspect, the present technology provides methods, compounds, and compositions for treating glucose intolerance and/or diseases or disorders associated with glucose intolerance. In particular, the present technology provides a method for treating glucose intolerance and/or diseases or disorders associated with glucose intolerance in a mammalian subject in need thereof, comprising administering to the subject a therapeutically effective amount of one or more first compound that increases the level and/or activity of multidrug resistance protein 1 (MRP1), wherein the therapeutic amount of the first compound reduces migration of neutrophils into the target tissue, and/or administering a therapeutically effective amount of one or more second compound that inhibits one or more of multidrug resistance protein 2 (MRP2), and hepoxilin A3 (HXA₃) synthase, wherein the therapeutic amount of the second compound reduces migration of neutrophils into the target tissue, and/or administering a therapeutically effective amount of one or more third compound that increases one or more N-acylethanolamines (NAEs), wherein the therapeutic amount of the third compound reduces migration of neutrophils into the target tissue.

In one embodiment, the present disclosure provides methods for treating neutrophil-mediated inflammation by targeting the pro-inflammatory MRP2/HXA₃ pathway, comprising administering to the subject a therapeutically effective amount of one or more compound that inhibits the activity and/or level of one or more of multidrug resistance protein 2 (MRP2) and hepoxilin A3 (HXA₃) synthase, wherein the therapeutic amount of the compound reduces migration of neutrophils into the target tissue.

In another embodiment, the present disclosure also provides methods for treating neutrophil-mediated inflammation by targeting the anti-inflammatory P-gp/endocannabinoid pathway, comprising administering to the subject a therapeutically effective amount of one or more compound that increases the level and/or activity of one or more N-acylethanolamines (NAEs), wherein the therapeutic amount of the compound reduces migration of neutrophils into the target tissue.

In a further embodiment, the present disclosure further provides methods for treating neutrophil-mediated inflammation, comprising administering to the subject a therapeutically effective amount of one or more second compound that increases the level and/or activity of multidrug resistance protein 1 (MRP1), wherein the therapeutic amount of the compound reduces migration of neutrophils into the target tissue.

In yet another embodiment, the present disclosure provides methods for treating neutrophil-mediated inflammation by targeting both the anti-inflammatory P-gp/endocannabinoid, and the pro-inflammatory MRP2/HXA₃ pathway, the method comprising administering to the subject a therapeutically effective amount of (A) one or more first compound that inhibits the activity and/or level of one or more of multidrug resistance protein 2 (MRP2) and hepoxilin A3 (HXA₃) synthase, and (B) one or more second compound that increases the level and/or activity of one or more N-acylethanolamines (NAEs), wherein the therapeutic amount of the first and second compounds reduces migration of neutrophils into the target tissue.

### III. Compounds of the Present Technology

The present technology provides compositions for treating glucose intolerance and conditions associated therewith. In some embodiments, the present technology provides compositions comprising one or more of a first compound that increases the level and/or activity of multidrug resistance protein 1 (MRP1), a second compound that inhibits one or more of multidrug resistance protein 2 (MRP2) and hepoxilin A3 (HXA₃) synthase, and/or a third compound that increases one or more N-acylethanolamines (NAEs).

The present technology provides compositions for treating neutrophil-mediated inflammation and conditions associated therewith. In some embodiments, the present technology provides compositions comprising one or more of a first compound that increases the level and/or activity of multidrug resistance protein 1 (MRP1), a second compound that inhibits one or more of multidrug resistance protein 2 (MRP2) and hepoxilin A3 (HXA₃) synthase, and/or a third compound that increases one or more N-acylethanolamines (NAEs).

In some embodiments, the present technology discloses a cyclosporine A-polymer conjugate and precursor thereof defined by Formula I:

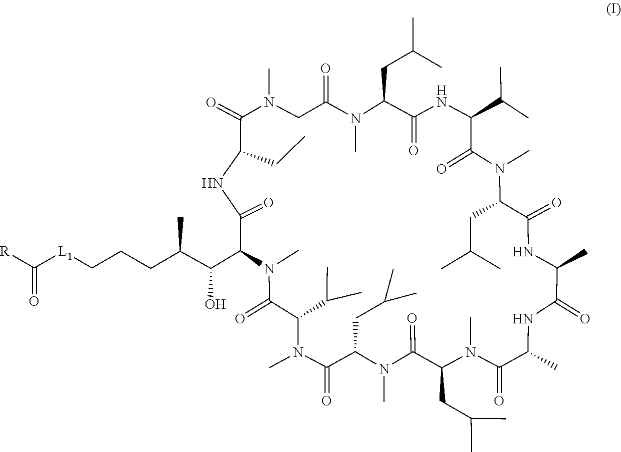

a stereoisomer thereof, or a pharmaceutically acceptable salt of any of the foregoing. In Formula I, L₁ may be a C₀ to C₄ alkyl group optionally substituted with one or more F. For example, in some embodiments, L₁ may be methylene, ethylene, propylene or butylene. In some embodiments, L₁ may be Co. In some embodiments, L₁ may be C₁-C₄ fluoroalky. The fluoroalkyl may have 1, 2, 3, 4 or more F and/or may be perfluorinated. In Formula I, R may be —OH or a polymer, optionally including a linker group, L₂.

In some embodiments, the polymer of R is selected from the group consisting of dextran, polyethylene glycol (PEG), periodate-oxidized dextran, polysialic acids (PSAs), hyaluronic acid (HA), dextrin, hydroxyethyl-starch (HES), poly(2-ethyl 2-oxazoline) (PEOZ), polyglutamic acid (PGA), polylactic acid (PLA), polylactic-co-glycolic (PLGA), poly(D,L-lactide-co-glycolide) (PLA/PLGA), poly(hydroxyalkylmethaacrylamide), polyglycerol, 25 polyamidoamine (PAMAM), polyethylenimine (PEI), and polypeptides. In some embodiments, the polymer is PEG. The PEG polymers may be functionalized with amine (NH₂) and/or aldehyde (CHO) that include linear mono-amines and mono-aldehydes, linear bi-amines and bi-aldehydes, multi-arm-amines and multi-arm-aldehydes, branched mono-, bi- and multi-armed-amines and aldehydes and multi-arm-forked-amines and aldehydes. In some embodiments, the polymer is PEG having 40-50 ethylene oxide subunits, i.e., 40, 41, 42, 43, 44, 45, 46, 47, 48, 49 or 50 ethylene oxide subunits or a range between and including any two of the foregoing values. The polymers can be of any molecular weight as described herein.

In some embodiments, the polymer has an average molecular weight in the range of about 100 Da to about 800 kDa. (Unless otherwise indicated, “average molecular weight” means weight average molecular weight.) In some embodiments the polymer has an average molecular weight in the range of about 1 kDa to about 800 kDa. In some embodiments, the polymer has an average molecular weight less than 1 kDa. In some embodiments, the polymer has an average molecular weight less than 10 kDa. In some embodiments, the average molecular weight of the polymer is about 1 kDa, 2 kDa, 5 kDa, 10 kDa, 20 kDa, 30 kDa, 40 kDa, 50 kDa, 60 kDa, 70 kDa, 80 kDa, 90 kDa, 100 kDa, 125 kDa, 150 kDa, 175 kDa, 200 kDa, 225 kDa, 250 kDa, 275 kDa, 300 kDa, 325 kDa, 350 kDa, 375 kDa, 400 kDa, 425 kDa, 450 kDa, 475 kDa, 500 kDa, 550 kDa, 600 kDa, 650 kDa, 700 kDa, 750 kDa, 800 kDa, or any range between and including two of these values.

The polymers described herein can have any of a number of different geometries. For example, in some embodiments, the polymers are linear polymers, branched polymers, forked polymers, or a combination of any of these polymers.

As noted above, the R group in the compound of Formula I (or IA) optionally includes a linker, L₂. In some embodiments, the linker L₂ is a biodegradable linker. In some embodiments, the biodegradable linker comprises an oligopeptide having from 2 to 10 amino acid residues. The residues may be selected from the naturally occurring amino acids.

In some embodiments, the linker L₂ comprises a substituted or unsubstituted C₁-C_{X} alkylene, cycloalkylene, cycloalkylalkylene, heteroalkylene, alkenylene, or heteroalkenylene group, wherein x may be any integer from 1 to 12, i.e., 1, 2, 3, 4, 5, 6, 7, 8, 9, 10, 11 or 12. For example, L₂ may comprise a C1-Cx fluoroalkyl group where one or more of the hydrogen atoms are fluorine atoms, such as 1, 2 or 3 or more fluorines. In some embodiments, L₂ may be a heteroalkylene containing one or two NH groups, including but not limited to (C₁-C₁₀ alkylene)—NH (e.g., CH₂CH₂NH, CH₂CH₂CH₂NH, CH₂CH₂CH₂CH₂NH, CH₂CH(CH₃)CH(CH₃)CH₂NH), (Cₙ alkylene)NH(Cₚ alkylene) where n, p are independently an integer from 1-10, but n+p does not exceed 10 (e.g., CH₂CH₂CH₂NHCH₂CH₂), NH-(C₁-C₁₀ alkylene)NH (e.g., NH(CH₂)sNH, NH(CH₂)₆NH, NH(CH₂)₈NH), or NH(Cₙ alkylene)NH(Cₚ alkylene) where n and p are integers as defined previously (e.g., NHCH₂CH₂CH₂NH CH₂CH₂, NH(CH₂)6NHCH₂). In some embodiments, the L₂ may be a heteroalkylene that contains one or two oxygen atoms, including but not limited to (C₁-C₁₀ alkylene)-O (e.g., CH₂CH₂O, CH₂CH₂CH₂₀, CH₂CH₂CH₂CH₂₀, CH₂CH(CH₃)CH(CH₃)CH₂O), (Cₙ alkylene)((Cₚ alkylene) where n, p are independently an integer from 1-10, but n+p does not exceed 10 (e.g., CH₂CH₂CH₂OCH₂CH₂), O-(C₁-C₁₀ alkylene)O (e.g., O(CH₂)₅O, O(CH₂)₆O, O(CH₂)80), or O(Cₙ alkylene)((Cₚ alkylene) where n and p are integers as defined previously (e.g., OCH₂CH₂CH₂₀ CH₂CH₂, O(CH₂)₆OCH₂). In some embodiments, L₂ may be a heteroalkylene containing an O and an NH group, including but not limited to NH—(C₁-C₁₀ alkylene)O, (e.g., NH(CH₂)₅O, NH(CH₂)₆O, NH(CH₂)₈O), or NH(Cₙ alkylene)O(Cₚ alkylene) where n and p are integers as defined previously (e.g., NHCH₂CH₂OCH₂CH₂, O(CH₂)₆NHCH₂).

In some embodiments of the compound of Formula I (including Formula IA) R is —OH or a PEG group having from 40 to 50 ethylene oxide units and includes a linker, L₂, that is a substituted or unsubstituted heteroalkylene group, e.g., an unsubstituted aminoalkylene group. In some embodiments, R is selected from —OH or —NH(CH₂)₂-6(CH₂CH₂O)₄₂₋₄₆—O(CH₂)₀₋₅CH₃. In some embodiments, R is selected from the group consisting of CH₃O-(CH₂ CH₂O)+-CH₂CH₂CH₂NH—(BT051), CH₃₀-(CH₂ CH₂O)₄₄—CH₂CH₂NH- (BT090), and HO—(BT070). In any such embodiments m may be 2 and have the structure of Formula IA.

The cyclosporine A-polymer conjugates and precursors may be prepared using standard techniques known in the art. In some embodiments, a difunctional linker containing at least two functional groups containing heteroatoms selected from N, O, and S in which one of the functional groups is protected may be conjugated using standard ester, thioester and amide bond forming technology. For example, a diamino-alkylene linker in which one of the amino groups is protected by a urethane protecting group (e.g., Boc. Cbz, etc.) may be coupled to cyclosporine A in the presence of a coupling agent (e.g., DCC, EDC/HOBt, etc.). Alternatively, an active ester, mixed anhydride or acid halide derivative of cyclosporine A may be prepared and reacted with the mono-protected diamine. (See, for example, Bodansky, M. & Bodanszky, A., The Practice of Peptide Synthesis, Springer-Verlag, New York, 1984.) The protecting group may be removed and the free amine reacted with an aldehyde derivative of the polymer under reducing conditions to provide the conjugate. Similarly, a linker with a protected aldehyde (e.g., 1,1- dimethoxy) and an amine may be coupled to the cyclosporine A, deprotected to form the aldehyde and subjected to reductive amination with an amino-bearing polymer to form the conjugate. Variations of these schemes using α-107 -carboxy amines, α-107 -aminoalcohols, α-107 -carboxyalcohols, α-107 -aminothiols, and the like to link cyclosporine A and the polymer will be readily understood by those of skill in the art.

In some embodiments, cyclosporine A compounds of the present technology are used in combination with one or more compounds that increase (multidrug resistance protein 1) MRP1 for the treatment of inflammatory disease.

In some embodiments, cyclosporine A compounds of the present technology are used in combination with one or more multidrug resistance protein 2 (MRP2) inhibitors for the treatment of inflammatory disease. In some embodiments, the MRP2 inhibitor is selected from the group consisting of MRP2 RNAi; 3-([3-(2-[7-chloro-2-quinolinyl]ethenyl)phenyl-(3-dimethylamino-3-oxopropyl)-thio-methyl]thio)propanoic acid (also known as “MK571” and CysLT1 (LTD4) leukotriene receptor inverse agonist) (Tocris, Minneapolis, USA) (Genuuso et al. (2004) PNAS 101:2470-2475); Probenecid (also known as “PROBALANTM”), exemplified by probenecid inhibition of MRP2; FUROSEMIDE®; RITONAVIR®; SAQUINAVIR®; LAMIVUDINE®; ABACAVIR®; EMTRICITABINE®; EFAVIRENZ®; DELAVIRDINE®; NEVIRAPINE®; CIDOFOVIR®; ADEFOVIR®; and TENOFOVIR®. In some embodiments, the compound is conjugated to a polymer.

In some embodiments, the compound that inhibits the MRP2 comprises one or more of a compound that inhibits Hepoxilin A3 synthase, such as Hepoxilin A3 synthase RNAi. In some embodiments, the compound is conjugated to a polymer.

In some embodiments, the compound that inhibits the MRP2 comprises one or more compounds that inhibit fatty acid amide hydrolase (FAAH), such as FAAH RNAi; FAAH Inhibitor I (PubChem CID: 295380) 4-phenylmethoxyphenyl) N-butylcarbamate); URB597(PubChem CID: 1383884) 3′-Carbamoyl-[1,1′-biphenyl]-3-yl cyclohexylcarbamate; FAAH inhibitor 1 (PubChem CID: 1190414) N-(4-(6-methylbenzo[d]thiazol-2-yl)phenyl)-1-(thiophen-2-ylsulfonyl)piperidine-4-carboxamide; FAAH Inhibitor, 21 (PubChem CID:71699786); FAAH Inhibitor, 2i (PubChem CID: 71699785) N-Cyclohexylcarbamic acid 4-(dimethylamino)-3-phenylphenyl ester; FAAH Inhibitor, 2h (PubChem CID: 71699784) N-Cyclohexylcarbamic acid 4-(hydroxymethyl)-3-phenylphenyl ester; FAAH Inhibitor, 2j (PubChem CID: 58801136); FAAH Inhibitor, 2e (PubChem CID: 58801135); FAAH Inhibitor, 2a (PubChem CID: 58801134); FAAH Inhibitor, 2b (PubChem CID: 58801129); FAAH Inhibitor, 2f (PubChem CID: 58801126) Carbamic acid, cyclohexyl-, 6-methyl[1,1′-biphenyl]-3-yl ester; FAAH Inhibitor, 2k (PubChem CID: 58801125); FAAH Inhibitor, 2c (PubChem CID: 57582480); FAAH Inhibitor, 2g (PubChem CID: 44626363); FAAH Inhibitor, 2d (PubChem CID: 44626362); AM374, palmitylsulfonyl fluoride; ARN2508, derivative of flurbiprofen; BIA 10-2474; BMS-469908; CAY-10402; JNJ-245; JNJ-1661010; JNJ-28833155; JNJ-40413269; JNJ-42119779; JNJ-42165279; LY-2183240; Cannabidiol; MK-3168; MK-4409; MM-433593; OL-92; OL-135; PF-622; PF-750; PF-3845; PF-04457845; PF-04862853; RN-450; SA-47; SA-73; SSR-411298; ST-4068; TK-25; URB524; URB597 (KDS-4103, Kadmus Pharmaceuticals); URB694; URB937; VER-156084; V-158866; and Multiple FAAH inhibitors from ChemCruz® Biochemicals, Dallas, Texas). In some embodiments, the compound is conjugated to a polymer.

In some embodiments, the compound that inhibits the MRP2 comprises one or more compounds that inhibit P-glycoprotein (P-gp), such as P-gp RNAi; SipA; and small molecules (e.g., zosuquidar trihydrochloride (LY335979); VALSPODAR® (PSC₈₃₃) (Inhibitor of P-gp-mediated MDR); CP 100356 hydrochloride (Sigma-Aldrich); and Elacridar hydrochloride (R&D Systems). See also, WO 2004071498 A1; WO 2014106021 A1; WO 2005033101 A1; WO 2004009584 A1; WO 2002030915 A2; US 20100029755 A1; and US 20060073196 A1). In some embodiments, the compound is conjugated to a polymer.

In some embodiments, cyclosporine A compounds of the present technology (including but not limited to compounds of Formulas I and IA) are used in combination with one or more compounds that increase N-acylethanolamines (NAEs) for the treatment of inflammatory disease. In some embodiments, the compound that increases NAEs is a cannabinoid receptor type 2 (CB2) “agonist” (i.e., a compound that specifically binds to, and activates, CB2). Illustrative CB2 agonists include GW-405,833; AM-1241; HU-308; JWH-015; JWH-133; L-759,633; L-759,656; beta-caryophyllene; arachidonylcyclopropylamide; and arachidonyl-2′-chloroethylamide. In some embodiments, the compound is conjugated to a polymer.

In some embodiments, cyclosporine A compounds of the present technology are used in combination with one or more compounds that improve glucose tolerance, such as metformin; incretin mimetics; DPP-4-resistant GLP-1 receptor agonists, such as exenatide and liraglutide, DPP-4 inhibitors (sitagliptin, vildagliptin); insulin; amylin; pramlintide acetate; oral hypoglycemic drugs; and orlistat, or any combination thereof. In some embodiments, the method further comprises administering one or more In some embodiments, the compound is conjugated to a polymer.

### IV. Use of the Compositions of the Present Technology

The present technology provides methods for treating, preventing, or ameliorating glucose intolerance and/or diseases or conditions associated with glucose intolerance in a mammalian subject in need thereof, or neutrophil-mediated inflammation in a target tissue of a mammalian subject in need thereof, comprising administering to the subject a therapeutically effective amount of one or more first compound that inhibits one or more of N-formyl peptide receptor 1 (FPR1), multidrug resistance protein 2 (MRP2), and hepoxilin A3 (HXA₃) synthase, wherein the therapeutically effective amount of the first compound improves glucose tolerance and/or reduces migration of neutrophils into the target tissue. In some embodiments, the first compound is a cyclosporine A conjugate or precursor thereof such as, e.g., compounds of Formulas I or IA. In some embodiments, the method further comprises administering to the subject a therapeutically effective amount of one or more second compound that increases one or more N-acylethanolamines (NAEs), wherein the therapeutic amount of the second compound reduces migration of neutrophils into the target tissue. In a further embodiment, the method further comprises administering to the subject a therapeutically effective amount of one or more second and/or third compound that increases the level and/or activity of multidrug resistance protein 1 (MRP1), wherein the therapeutic amount of one or more second and/or third compound reduces migration of neutrophils into the target tissue. In another embodiment, the compounds of the present technology are administered singly or in any combination to a topical surface of the target tissue and/or at a luminal surface of the target tissue. In a further embodiment, the first compound that reduces migration of neutrophils into the target tissue is conjugated to a polymer. In another embodiment, the inflammation is non-infectious and/or infectious inflammation.

The present technology also provides methods for treating, ameliorating, or preventing neutrophil-mediated inflammation in a target tissue of a mammalian subject in need thereof, comprising administering to the subject a therapeutically effective amount of one or more first compound that increases one or more N-acylethanolamines (NAEs), wherein the therapeutic amount of the first compound reduces migration of neutrophils into the target tissue. In one embodiment, the method further comprises administering to the subject a therapeutically effective amount of one or more second compound that inhibits one or more of multidrug resistance protein 2 (MRP2) and HXA₃ synthase, wherein the therapeutic amount of the second compound reduces migration of neutrophils into the target tissue. In some embodiments, the second compound is a cyclosporine A conjugate or precursor thereof such as, e.g., compounds of Formulas I or IA. In another embodiment, the method further comprises administering to the subject a therapeutically effective amount of one or more second and/or third compound that increases the level and/or activity of multidrug resistance protein 1 (MRP1), wherein the therapeutic amount of the one or more second and/or third reduces migration of neutrophils into the target tissue. In a further embodiment, the one or more first compound that increases the one or more NAEs is a cannabinoid receptor type 2 (CB2) agonist. In another embodiment, the first compound that reduces migration of neutrophils into the target tissue is conjugated to a polymer.

In one aspect, the methods, compounds, and compositions of the present technology relate to cyclosporine A-polymer conjugates and precursors defined by Formula I as well as stereoisomers thereof, and pharmaceutically acceptable salts of any of the foregoing:

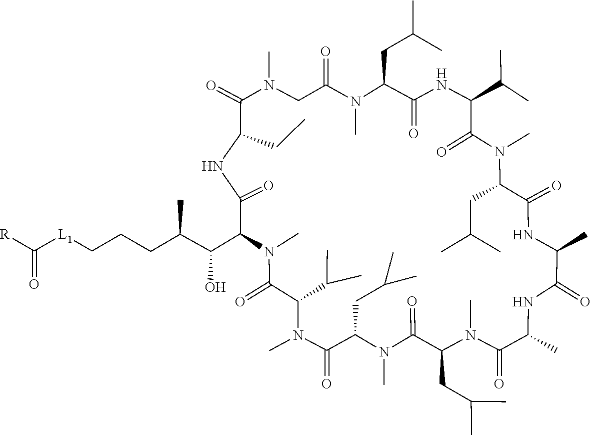

wherein L₁ and R may be as defined herein, as well as the use of one or more of these compounds (including but not limited to Formula IA, BT051 (also referred to as BT-051), BT090 (also referred to as BT-090), BT122, BT123, BT125, and BT126) to treat, ameliorate, or prevent glucose intolerance and/or diseases or conditions associated with glucose intolerance in a subject in need thereof, or neutrophil-mediated inflammation in a target tissue in a subject in need thereof. In other embodiments, the compounds of Formulas I and IA, in combination with one or more compounds (e.g., a compound that increases the level and/or activity of MRP1, or a compound that increases NAEs) will show a synergistic effect in this regard.

In some embodiments, the methods, compounds, and compositions of the present technology relate to the use of one or more of the cyclosporine A compounds of Formula I and IA to treat, ameliorate, or prevent glucose intolerance and/or diseases or conditions associated with glucose intolerance, such as type 2 diabetes mellitus, type 1 diabetes mellitus, gestational diabetes mellitus, neonatal diabetes, maturity-onset diabetes of the young, drug-induced diabetes, chemical-induced diabetes, hyperglycemia, impaired glucose tolerance (IGT), impaired fasting glucose (IFT), obesity, insulin resistance, metabolic syndrome, asthma, chronic obstructive pulmonary disease (COPD), Alzheimer's disease, cancers, Klinefelter Syndrome, Turner Syndrome, Wolfram Syndrome, Cystic Fibrosis, Friedreich's ataxia, multiple sclerosis, down syndrome, muscular dystrophy, polycystic ovary syndrome (PCOS), uremia, cirrhosis, chronic renal failure, and HAART-treated HIV infection.

The methods of the present technology are useful for treating “inflammation,” which is a physical condition in which part of the body reacts to injury and/or infection. The classic symptoms of inflammation are heat, redness, swelling, pain, and/or loss of function. These are manifestations of the physiologic changes that occur during the inflammatory process. The three major components of this process are: (1) changes in the caliber of blood vessels and the rate of blood flow through them (hemodynamic changes); (2) increased capillary permeability; and (3) leukocytic exudation. “Neutrophil-mediated inflammation” refers to the leukocytic exudation and stage of inflammation, in which neutrophils move to the endothelial lining of the small blood vessels (margination) and line the endothelium in a tightly packed formation (pavementing). Eventually, these neutrophils move through the endothelial spaces and escape into the extravascular space (emigration). Once they are outside the blood vessels they are free to move and, by chemotaxis, are drawn to the site of injury. Accumulations of neutrophils (and macrophages) at the area of inflammation act to neutralize foreign particles by phagocytosis.

Inflammation includes acute inflammation, which is usually of sudden onset, marked by the classical signs of heat, redness, swelling, pain, and loss of function, and in which vascular and exudative processes predominate; catarrhal inflammation, which is a form affecting mainly a mucous surface, marked by a copious discharge of mucus and epithelial debris; chronic inflammation, which is prolonged and persistent inflammation marked chiefly by new connective tissue formation; it may be a continuation of an acute form or a prolonged low-grade form; interstitial inflammation, which is inflammation affecting chiefly the stroma of an organ; traumatic inflammation, which is one that follows a wound or injury; ulcerative inflammation, in which necrosis on or near the surface leads to loss of tissue and creation of a local defect (ulcer).

Inflammation may be infectious and/or non-infectious. “Infectious” inflammation refers to inflammation that is associated with and/or is caused by the invasion and multiplication of microorganisms such as bacteria, viruses, and parasites that are not normally present within the body. In contrast, “non-infectious” inflammation refers to inflammation that is not associated with and/or is not caused by the invasion and multiplication of microorganisms such as bacteria, viruses, and parasites that are not normally present within the body.

In another embodiment, the present technology provides a method for treating neutrophil-mediated inflammation by targeting the pro-inflammatory MRP2/HXA₃ pathway. In a particular embodiment, this method for treating neutrophil-mediated inflammation in a target tissue of a mammalian subject in need thereof, comprises administering to the subject a therapeutically effective amount of one or more first compound that inhibits the activity and/or level of one or more of multidrug resistance protein 2 (MRP2) and hepoxilin A3 (HXA₃) synthase, wherein the therapeutic amount of the first compound reduces migration of neutrophils into the target tissue. In some embodiments, the compound is a cyclosporine A conjugate or precursor, such as a compound of Formula I (including but not limited to Formula IA, BT051, BT090, BT122, BT123, BT125, and BT126).

In various embodiments, suitable in vitro or in vivo assays are performed to determine the effect of a specific composition of the present technology and whether its administration is indicated for treatment. In various embodiments, in vitro assays can be performed with representative cell-based assays, such as the neutrophil migration assay. In other embodiments, in vivo models, typified by animal models, may be used to determine if a given cyclosporine A conjugate (or precursor) alone or in combination with one or more additional compounds (e.g., an additional compound that inhibitis one or more of MRP2 nd HXA₃ synthase, a compound that increases the level and/or activity of MRP1, or a compound that increases NAEs), exerts the desired effect in treating a disease or condition. Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

In some embodiments, the methods of the present technology further comprise administering one or more antibiotic and/or anti-inflammatory agent. Examples of antibiotic/anti-inflammatory agents used singly or in combination in the methods of the present technology include, but are not limited to Dalbavancin (DALVANCE©, XYDALBA©), Oritavancin (ORBACTIVE©) Daptomycin (Cubicin©), Tedizolid (SIVEXTRO©), Ceftobiprole (ZEVTERA©, MABELIO©), Ceftolozane-tazobactam (ZERBAXA©) mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, steroidal agents such as corticosteroids such as hydrocortisone, hydroxyltriamcinolone alphamethyl dexamethasone, dexamethasonephosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, non-steroidal agents such as COX inhibitors, LOX inhibitors, p38 kinase inhibitors, immunosuppresant agents such as cyclosporin, and cytokine synthesis inhibitors, tetracycline, minocycline, and doxycycline, or any combination thereof.

For example, in subjects with glucose intolerance, it may be desirable that any one of the methods of the present technology further comprise administering one or more pharmacologic therapies such as metformin; incretin mimetics; DPP-4-resistant GLP-1 receptor agonists, such as exenatide and liraglutide, which are incretin mimetics; as well as the DPP-4 inhibitors sitagliptin and vildagliptin; insulin; amylin; pramlintide acetate; oral hypoglycemic drugs; and orlistat. Other therapies include lifestyle modification through diet (e.g., low-calorie, low-fat diet) and exercise.

### V. Combination Therapies

In some embodiments, the cyclosporine A compounds of the present technology may be combined with one or more additional therapeutic agents for the prevention, amelioration, or treatment of a disease or condition.

In one embodiment, an additional therapeutic agent is administered to a subject in combination with a cyclosporine A conjugate or precursor of the present technology (e.g., a compound of Formula I, including but not limited to Formula IA, BT051, BT090, BT122, BT123, BT125, and BT126) such that a synergistic therapeutic effect is produced.

In some embodiments, the cyclosporine A compounds of the present technology (e.g., a compound of Formula I, including but not limited to Formula IA, BT051, BT090, BT122, BT123, BT125, and BT126) are combined with one or more methods or compounds for the treatment or prevention glucose intolerance and/or diseases or conditions associated with glucose intolerance. In some embodiments, the one or more methods comprises a lifestyle modification such as a reduced calorie or reduced fat diet and/or exercise regimen. In some embodiments, it may be desirable that any one of the methods of the present technology further comprise administering one or more pharmacologic therapies such as metformin; incretin mimetics; DPP-4-resistant GLP-1 receptor agonists, such as exenatide and liraglutide, which are incretin mimetics; as well as the DPP-4 inhibitors sitagliptin and vildagliptin; amylin; pramlintide acetate; oral hypoglycemic drugs; orlistat, insulin; insulin degludec; insulin detemir; human insulin; biphasic human insulin; insulin aspart; biphasic insulin aspart; glucagon-like peptide-1; semaglutide; repaglinide; or combinations thereof.

In some embodiments, the cyclosporine A compounds of the present technology (e.g., a compound of Formula I, including but not limited to Formula IA, BT051, BT090, BT122, BT123, BT125, and BT126) are combined with one or more compounds that reduce levels of N-formyl peptides such as fMLP.

In some embodiments, the cyclosporine A compounds of the present technology (e.g., a compound of Formula I, including but not limited to Formula IA, BT051, BT090, BT122, BT123, BT125, and BT126) are combined with one or more compounds that increase levels of multidrug resistance protein 1 (MRP1) described above.

In some embodiments, the cyclosporine A compounds of the present technology (e.g., a compound of Formula I, including but not limited to Formula IA, BT051, BT090, BT122, BT123, BT125, and BT126) are combined with one or more additional compounds that inhibit one or more of multidrug resistance protein 2 (MRP2) and hepoxilin A3 (HXA₃) synthase described above.

In some embodiments, the cyclosporine A compounds of the present technology (e.g., a compound of Formula I, including but not limited to Formula IA, BT051, BT090, BT122, BT123, BT125, and BT126) are combined with one or more additional compounds that increase N-acylethanolamines (NAEs) described above.

In some embodiments, the cyclosporine A compounds of the present technology (e.g., a compound of Formula I, including but not limited to Formula IA, BT051, BT090, BT122, BT123, BT125, and BT126) are combined with one or more additional therapeutic agents for treating glucose intolerance and conditions associated therewith, including, but not limited to, type 2 diabetes mellitus, type 1 diabetes mellitus, gestational diabetes mellitus, neonatal diabetes, maturity-onset diabetes of the young, drug-induced diabetes, chemical-induced diabetes, hyperglycemia, impaired glucose tolerance (IGT), impaired fasting glucose (IFT), obesity, insulin resistance, metabolic syndrome, asthma, chronic obstructive pulmonary disease (COPD), Alzheimer's disease, cancers, Klinefelter Syndrome, Turner Syndrome, Wolfram Syndrome, Cystic Fibrosis, Friedreich's ataxia, multiple sclerosis, down syndrome, muscular dystrophy, polycystic ovary syndrome (PCOS), uremia, cirrhosis, chronic renal failure, and HAART-treated HIV infection. In some embodiments, the present technology provides compositions comprising one or more of a first compound that reduces the level of fMLP, reduces the activation of FPR1, increases the level and/or activity of multidrug resistance protein 1 (MRP1), a second compound, such as a cyclosporine A conjugate, that inhibits one or more of multidrug resistance protein 2 (MRP2) and hepoxilin A3 (HXA₃) synthase, and/or a third compound that increases one or more N-acylethanolamines (NAEs).

The multiple therapeutic agents (e.g., cyclosporine A conjugates, compounds that increase the level and/or activity of MRP1, additional inhibitors of MRP2 and HXA₃ synthase, and/or compounds that increase NAEs) may be administered in any order or even simultaneously. If simultaneously, the multiple therapeutic agents may be provided in a single, unified form, or in multiple forms (by way of example only, either as a single formulation or as two separate formulations). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

In some embodiments, the methods of the present technology further comprise administering to the subject a therapeutically effective amount of at least one compound that reduces the level of fMLP, reduces the activation of FPR1, increases the level and/or activity of one or more N-acylethanolamines (NAEs), wherein the therapeutic amount of the compound reduces migration of neutrophils into the target tissue.

In some embodiments, the compound that increases NAEs is a cannabinoid receptor type 2 (CB2) “agonist” (i.e., a compound that specifically binds to, and activates, CB2). CB2 agonists are exemplified by GW-405,833; AM-1241; HU-308; JWH-015; JWH-133; L-759,633; L-759,656; beta-caryophyllene; arachidonylcyclopropylamide; and arachidonyl-2′-chloroethylamide.

In some embodiments, the methods of the present technology may further comprise administering one or more antibiotic and/or anti-inflammatory agent. Examples of antibiotic/anti-inflammatory agents used singly or in combination in the methods of the present technology include, but are not limited to Dalbavancin (DALVANCE©, XYDALBA©), Oritavancin (ORBACTIVE©) Daptomycin (Cubicin©), Tedizolid (SIVEXTRO©), Ceftobiprole (ZEVTERAC, MABELIO©), Ceftobiprole (ZEVTERA©, MABELIO©), Ceftolozane-tazobactam (ZERBAXA©) mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, steroidal agents such as corticosteroids such as hydrocortisone, hydroxyltriamcinolone alphamethyl dexamethasone, dexamethasonephosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, non-steroidal agents such as COX inhibitors, LOX inhibitors, p38 kinase inhibitors, immunosuppresant agents such as cyclosporin, and cytokine synthesis inhibitors, tetracycline, minocycline, and doxycycline, or any combination thereof.

### VI. Modes of Administration

Any method known to those in the art for contacting a cell, organ, or tissue with compounds of the present technology may be employed. Suitable methods include in vitro, ex vivo, or in vivo methods.

In vitro methods typically include cultured samples. For example, a cell can be placed in a reservoir (e.g., tissue culture plate), and incubated with a compound under appropriate conditions suitable for obtaining the desired result. Suitable incubation conditions can be readily determined by those skilled in the art.

Ex vivo methods typically include cells, organs or tissues removed from a mammal, such as a human. The cells, organs or tissues can, for example, be incubated with the compound under appropriate conditions. The contacted cells, organs or tissues are typically returned to the donor, placed in a recipient, or stored for future use. Thus, the compound is generally in a pharmaceutically acceptable carrier.

In vivo methods typically include the administration of a compound of the present technology to a mammal such as a human. When used in vivo for therapy, a compound of the present technology is administered to a mammal in an amount effective to obtain the desired result, e.g., of treating the mammal. The effective amount is determined during pre-clinical trials and clinical trials by methods familiar to physicians and clinicians. The dose and dosage regimen will depend upon the degree of the disease or condition in the subject, the characteristics of the particular compound of the present technology used, e.g., its therapeutic index, the subject, and the subject's history.

An effective amount of a compound of the present technology useful in the present methods, such as in a pharmaceutical composition or medicament, may be administered to a mammal in need thereof by any of a number of well-known methods for administering pharmaceutical compositions or medicaments. The compounds of the present technology may be administered systemically or locally.

The compounds of the present technology described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

In some embodiments, the pharmaceutical compositions of the present disclosure contain a pharmaceutically acceptable carrier and/or excipient suitable for rendering the compound or mixture administrable orally as a tablet, capsule or pill, or parenterally, intravenously, intradermally, intramuscularly or subcutaneously, or transdermally.

Pharmaceutical compositions are typically formulated to be compatible with the intended route of administration. Administering the pharmaceutical composition of the present disclosure may be accomplished by any means known to the skilled artisan. Routes of administration include, but are not limited to, parenteral, intravenous, intramuscular, intradermal, intraperitoneal, intratracheal, subcutaneous, oral, intranasal/respiratory (e.g., inhalation), transdermal (topical), sublingual, ocular, vaginal, rectal, and transmucosal administration. Systemic routes include oral and parenteral. Several types of devices are regularly used for administration by inhalation. These types of devices include metered dose inhalers (MDI), breath-actuated MDI, dry powder inhaler (DPI), spacer/holding chambers in combination with MDI, and nebulizers.

For oral administration, the compounds can be formulated readily by combining the active compound(s) with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In some embodiments, administration is topical and/or at the luminal surface of the tissue to be treated. “Topical” administration of a composition means contacting the composition with the skin. “Luminal surface” refers to the inner open space or cavity of a tubular organ, such as the interior central space in an artery or vein through which blood flows; the interior of the gastrointestinal tract; the pathways of the bronchi in the lungs; the interior of renal tubules and urinary collecting ducts; the pathways of the female genital tract, starting with a single pathway of the vagina, splitting up in two lumina in the uterus, both of which continue through the fallopian tubes.

In some embodiments, the compounds of the present technology are administered topically and/or at a luminal surface of the target tissue. This is advantageous to reduce potential systemic toxic side effects of the compounds.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the disclosure is contained in a form within a matrix such as those described in U.S. Pat. Nos. 4,452,775, 4,675, 189, and 5,736, 152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

### EXPERIMENTAL EXAMPLES

The present technology is further illustrated by the following examples, which should not be construed as limiting in any way.

### Example 1: Synthesis of PEG-Cyclosporine A Conjugates

Illustrative examples of the general synthesis of PEG-cyclosporine A conjugates is shown in Schemes 1 and 2. As will be understood by those of skill in the art, the side chain to which PEG is attached may be varied in length by using suitable homologs of olefinic ester, 1. For example, benzyl pent-4-enoate may be substituted with benzyl prop-2-enoate (benzyl acrylate), benzyl but-3-enoate, benzyl hex-5-enoate, or benzyl hept-6-enoate to provide a shorter or longer side chain. The use of such homologs is within the skill in the art.

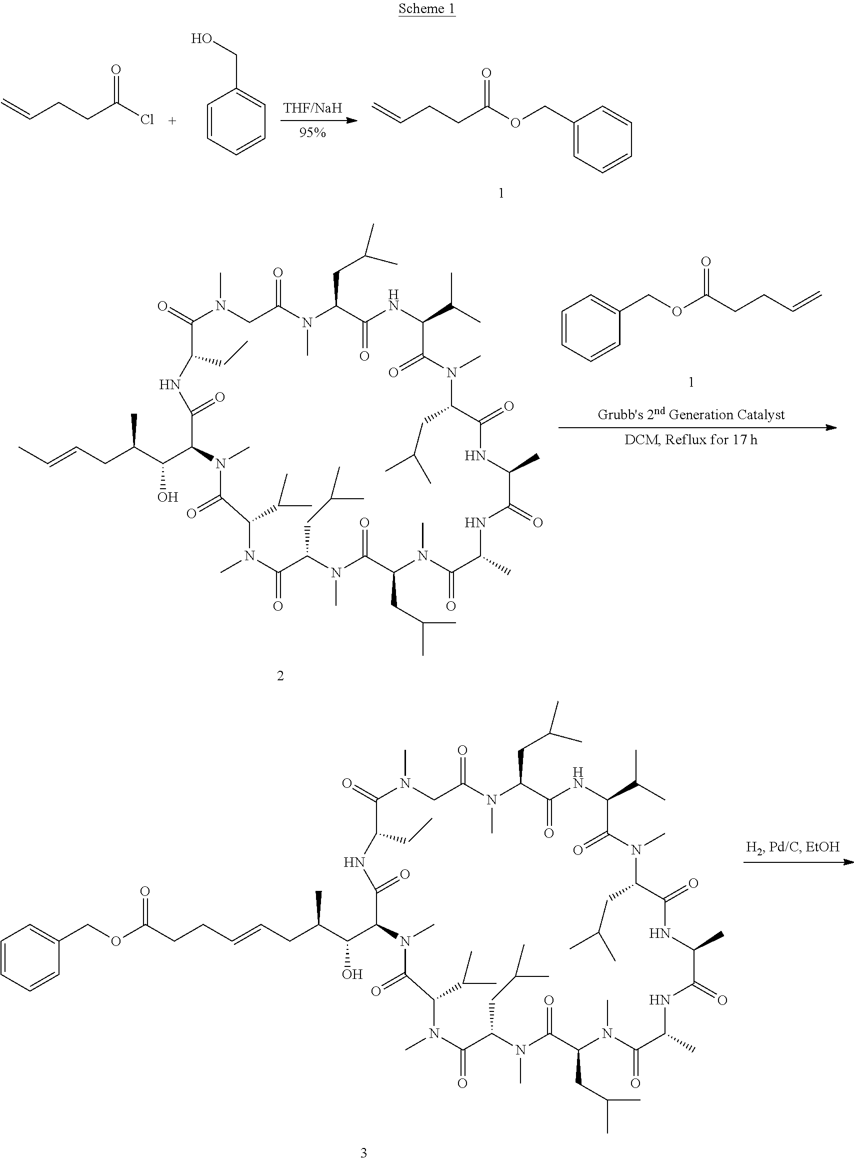

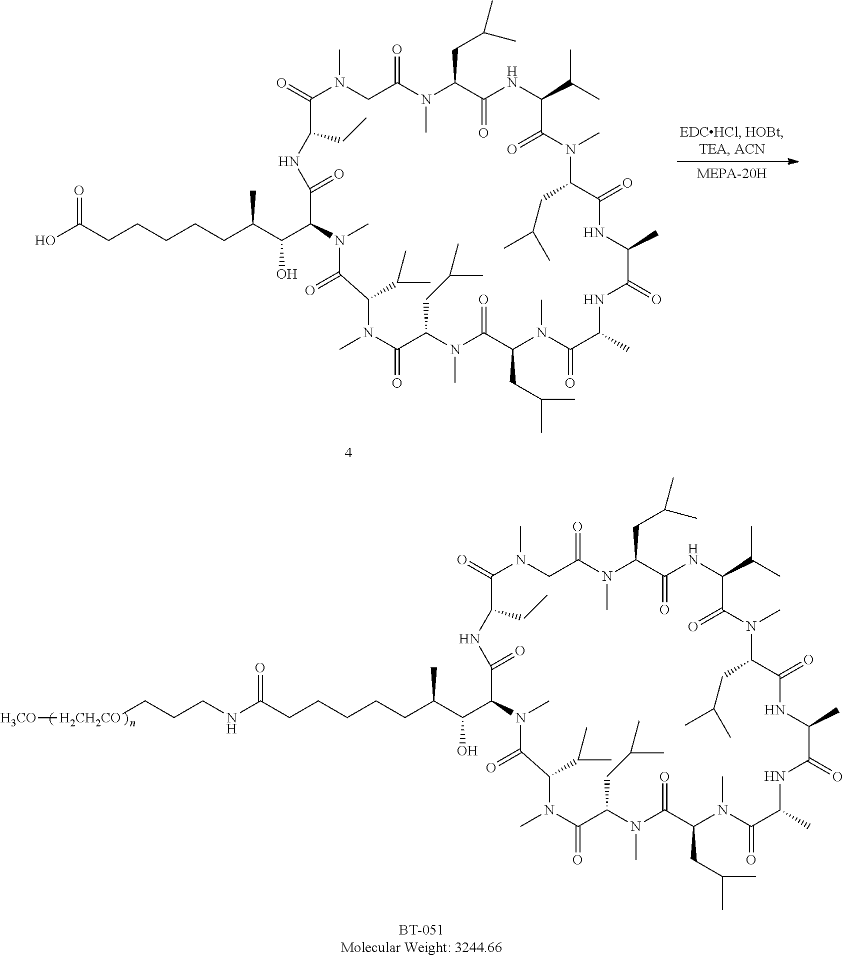

Synthesis of Intermediate 1. A 2 L round bottom flask equipped with a mechanical stirrer and a thermocouple was purged with nitrogen. Sodium hydride (18.9 g, 1.05 eq, 60% dispersion in mineral oil, Sigma) followed by anhydrous THF (540 mL, 10 vol, Sigma) was added. The mixture was cooled to <10 ° C. in an ice-water bath. Then, benzyl alcohol (50.9 mL, 0.473 mol, 1.05 eq, Sigma) was added dropwise over a period of 45 min. The internal temperature was maintained at <10 ° C.during the addition. The ice bath was removed and the reaction mixture was warmed to ambient temperature and was stirred for 15 min. The mixture was again cooled to <10 ° C. 4-pentenoyl chloride (50 mL, 1 eq, 0.450 mol, Sigma) was added dropwise over a period of 60 min (internal temperature went from 5 to 15 ° C.during the addition). The reaction was warmed to ambient temperature and was stirred for 20 h. The reaction was quenched with 30 mL sat. NH₄Cl and EtOAc (250 mL) was added. The layers were separated and organic layer was washed with sat. NaHCO₃ (200 mL), and then brine (200 mL). The organic layer was dried over anhydrous Na₂SO₄, filtered and the filtrate was concentrated by rotary evaporation to obtain crude 1 as an oil. The crude was purified by chromatography (SiO₂, 1 kg, 25 cm×10 cm, 10% EtOAc/heptanes) to give intermediate 1 (81.9 g, 95% yield) as a clear liquid. The ¹H NMR was consistent with the assigned structure and the HPLC purity was 98.9%.

Synthesis of Intermediate 3. A 25 mL round bottomed flask equipped with a magnetic stir bar was placed under a blanket of nitrogen. Compound 2 (1.40 g, 1.16 mmol, 1.0 eq) followed by DCM (35 mL, degassed by nitrogen purging) was added resulting in a solution. To this was added intermediate 1 (1.87 g, 9.84 mmol, 8.5 eq). This mixture was degassed by nitrogen purging for 5 min. The flask was then charged with Grubb's 2nd generation catalyst (0.11 g, 0.13 mmol, 0.13 eq, Sigma-Aldrich), and the mixture was again degassed with nitrogen purging for 5 min. The mixture was brought to reflux under argon with vigorous stirring for 17 h. The reaction mixture was cooled to ambient temperature and was evaporated to dryness. The residue was purified by chromatography (ISCO, SiO₂, 0-10% MeOH/DCM) to give intermediate 3 (1.1 g) as a light yellow solid, which contains 7.3% of compound 2 as determined by HPLC analysis.

The reaction was repeated on 5 g scale (compound 2) under the same conditions. The crude that was obtained will be purified by column chromatography.

Synthesis of Intermediate 4 (BT-070). A 150 mL Fisher Porter bottle was charged with intermediate 3 (1.0 g, 0.74 mmol, 1.0 eq), 10% Pd/C (0.40 g, 50% water, Johnson Matthey), and EtOH (20 mL). The flask was evacuated by vacuum and refilled with H₂ three times and was stirred under 50 PSI of hydrogen pressure for 23 h. The reaction mixture was filtered through a pad of Celite and the pad was washed with methanol (2×10 mL). The combined filtrate was concentrated by rotary evaporation to give intermediate 4 as a solid (0.95 g). As used herein, intermediate 4 of Scheme I also refers to BT-070.

Synthesis of BT051. A nitrogen flushed 50 mL R.B. flask equipped with a magnetic stir bar was charged with crude intermediate 4 (0.57, AMRI, JWU-B-5-1), MEPA-20H (1.00 g, NOF), HOBt·H₂O (0.096 g, Aldrich). The mixture was placed under a blanket of N₂, and was dissolved in acetonitrile (10 mL, Sigma Aldrich). TEA (0.18 mL) was added and the mixture was cooled in an ice-water bath. EDC.HCl (0.104 g, Sigma-Aldrich) was added in a single portion, and the reaction was removed from the ice bath and allowed to gradually warm to ambient temperature. The reaction was judged complete by HPLC analysis after 21 h. The reaction was diluted with DCM (50 mL) and was washed with DI H₂O (2×20 mL) and brine (20 mL). The organic layer was dried over anhydrous Na₂SO₄, filtered and the filtrate was concentrated under reduced pressure. The crude material was purified by chromatography (SiO₂, 5-15% MeOH/DCM) to give BT051 as a white solid (0.40 g, 27% yield). The ¹H NMR was consistent with the assigned structure and the HPLC purity was 98.2%.

The general synthesis of PEG-cyclosporine A conjugates as shown in Scheme 1 also led to the production of +/− CH₂ derivatives of BT-070 and BT051, which are shown below. Without wishing to be bound by theory, it is believed that the +/− CH₂ derivatives were generated by Scheme 1 due to the use of Grubb's catalyst in the first step of the synthesis, which encourages double bond migration. The derivative of BT-070 having one additional carbon in the linker (L1=—CH₂CH₂CH₂—) is BT125. The derivative of BT-070 having one fewer carbon in the linker (L1=—CH₂—) is BT122. The derivative of BT051 having one additional carbon in the linker (L1=—CH₂CH₂CH₂—) is BT126. The derivative having one fewer carbon in the linker (L1=—CH₂—) is BT123.

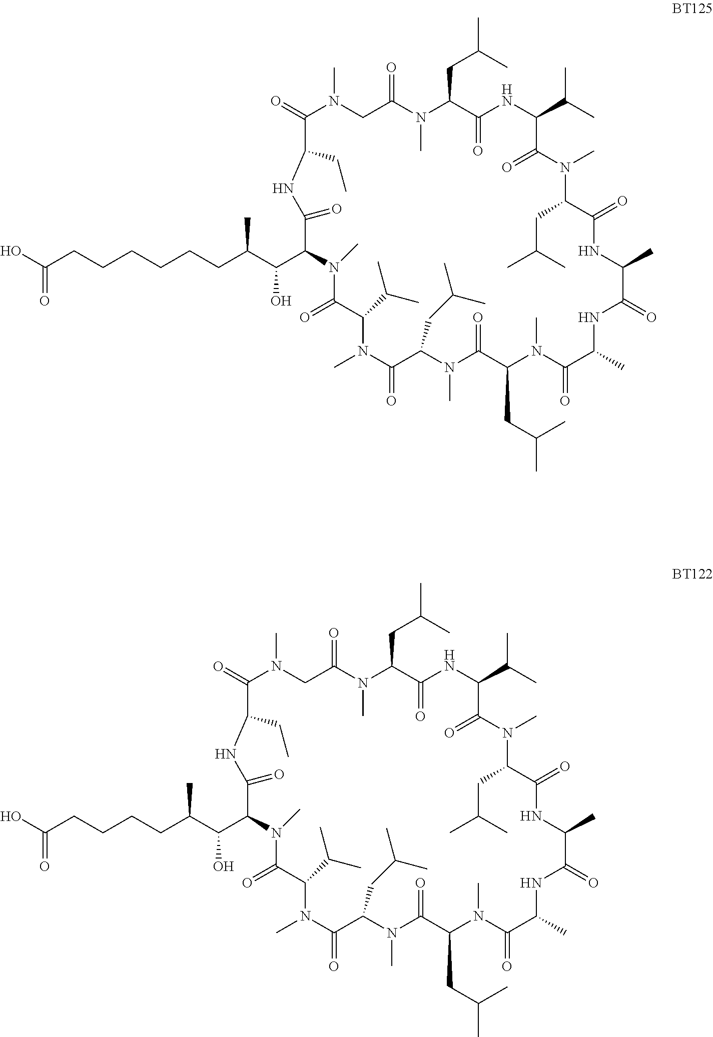 [SMILES: CC[C@@H]1NC(=O)[C@H]([C@H](O)[C@H](C)CCCCCCC(=O)O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O ; CC[C@@H]1NC(=O)[C@H]([C@H](O)[C@H](C)CCCCC(=O)O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

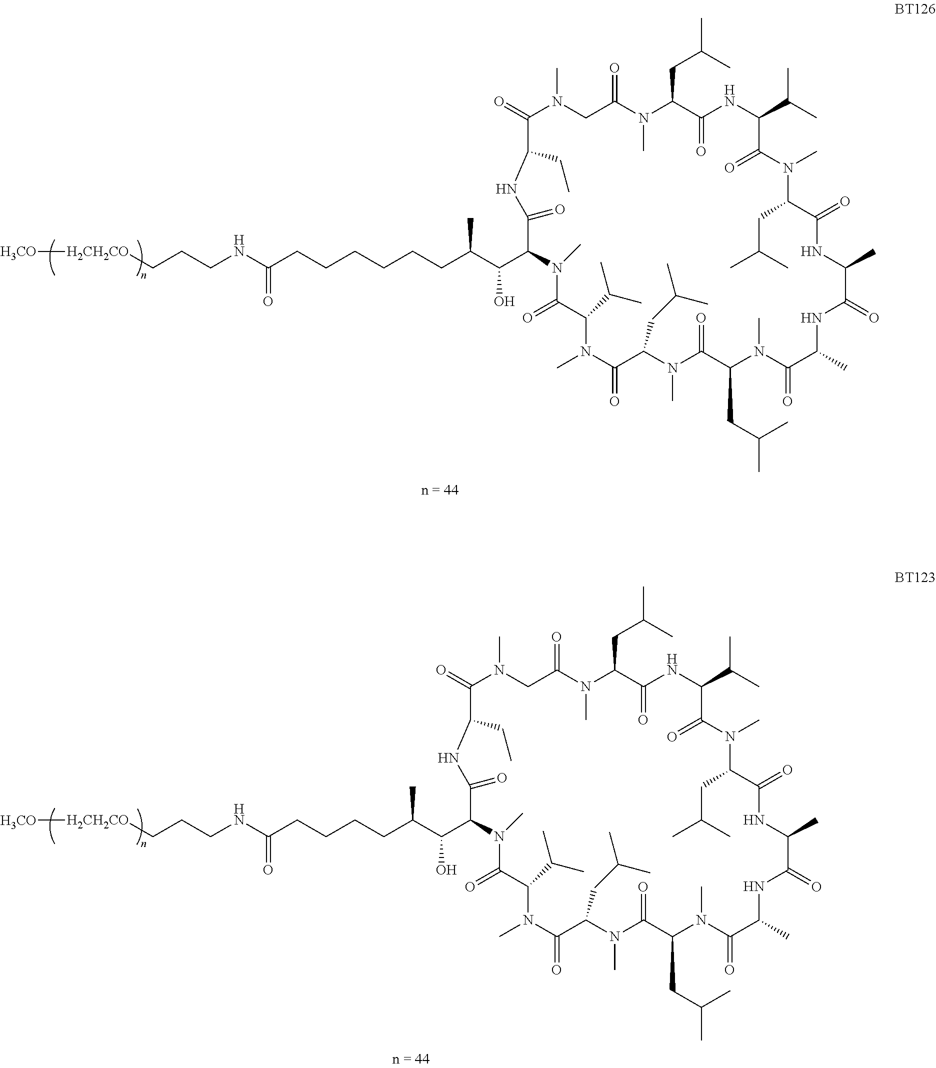

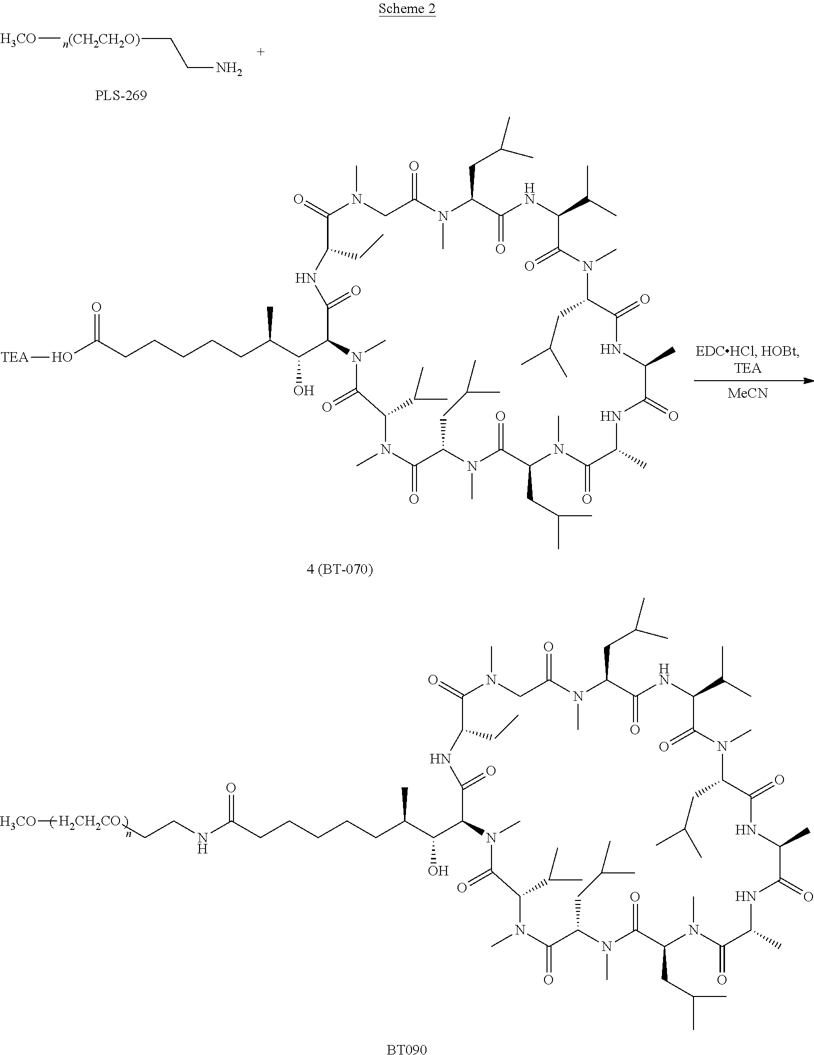

Synthesis of BT090. Reaction conditions: 1.0 eq CsA acid (BT070, aka Intermediate 4), 1.25 eq PLS-269, 1.4 eq EDC·HCl, 1.25 eq HOBt, 1 eq TEA, MeCN (4 mL, 31.7 vol), 0° C.-rt, 28.5 h (Scheme 2). To a 100 mL R.B. containing HOU-E-63-3 (0.126 g, AMRI) was charged MEPA-20H (0.230 g, NOF), HOBt·H₂O (0.019 g, Aldrich), and a stir bar. The reagents were placed under a blanket of N₂, dissolved in acetonitrile (3.2 mL, Sigma Aldrich) then TEA (0.013 mL, Aldrich), and were placed in an ice bath with stirring for 10 min. EDC·HCl (0.027 g, Sigma-Aldrich) was added in a single portion, and the reaction was removed from the ice bath and allowed to gradually warm to room temperature. After 21 h, the reaction was diluted with DCM (50 mL, Pride) and was extracted with DI H₂O (2×20 mL), brine (20 mL), dried over Na₂SO₄, and concentrated under reduced pressure. The crude material was purified by chromatography (SiO₂, 5-15% MeOH/DCM) to give product (0.181 g, 61%). The material was dissolved in a minimal amount water, frozen evenly in a dry ice/acetone bath, and lyophilized until dry to give a white solid.

### Example 2: Inhibition of Neutrophil Migration by Compounds of the Present Technology

This example demonstrates the efficacy of the compounds of the present technology in inhibiting neutrophil migration in vitro, and that the compounds of the present technology exhibit a dose-responsive inhibition of neutrophil migration.

PMN transmigration assay. T84 colorectal cancer cells were grown to confluence on the bottom side of 12 well polycarbonate membrane inserts of a 24 well Transwell® plate. The confluent growth of these epithelial cells results in physiologically-relevant apical (luminal) and basolateral surfaces in the transwells.

The apical surface of the monolayer was treated with BT051, BT070, BT090, BT122, BT123, BT125, or BT126 over a dose range for 1 hour. The monolayer was then infected on the apical surface with Salmonella typhimurium for 2 hours to induce hepoxilin A₃ (HXA₃) efflux through the MRP2 membrane protein and into the apical chamber.

The bacteria were washed off the monolayer and the apical surface re-exposed to BT051, BT070, BT090, BT122, BT123, BT125, or BT126. Freshly isolated and prepared human neutrophils were then added to the basolateral side and allowed to transmigrate through the monolayer for 2 hours. The number of neutrophils migrated to the apical chamber (due to the HXA₃ gradient) was then assayed by determination of myeloperoxidase activity (a neutrophil biomarker). Inhibition of transmigration is indicated by lower MPO activity in the apical chamber.

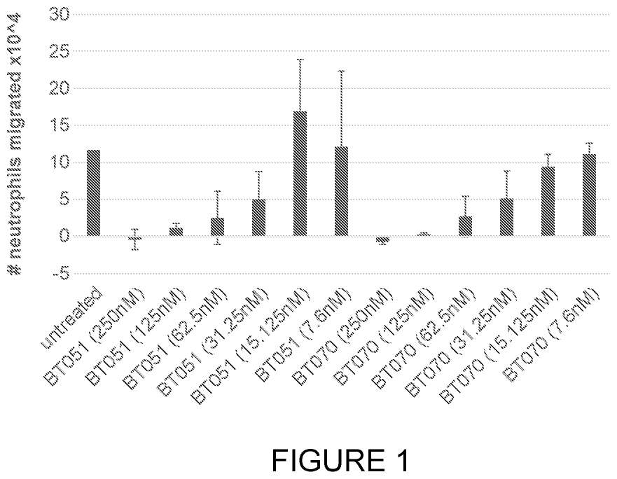

Results. As shown in FIG. 1, BT051 and BT070 exhibit dose-responsive inhibition of neutrophil migration. A 50% inhibition was observed at compound concentrations of 31.25 nM.

As shown in Table A, BT051 and BT070 exhibit inhibition of neutrophil migration at a dose of 100 nM. However, BT090 does not exhibit inhibition of neutrophil migration at a dose of 100 nM. For the purpose of Table A, the PEG-cyclosporine A conjugate/precursor is represented by the compound having the following formula:

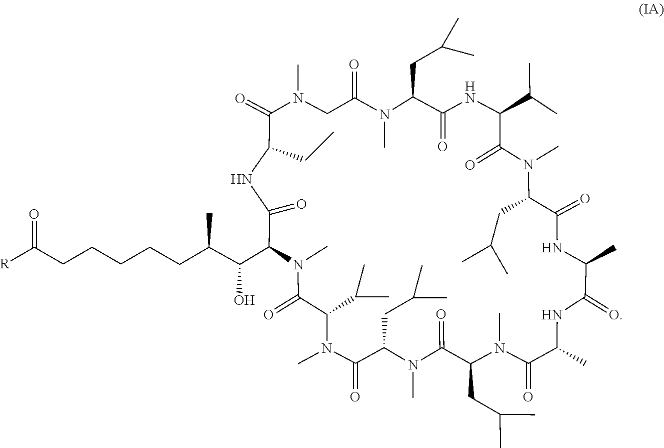

TABLE A
Inhibitory activity of PEG-cyclosporine A conjugates
and precursors in the neutrophil transmigration
assay (compounds tested at 100 nM).

| Compound | R═ | % Inhibition |
|---|---|---|
| BT051 | CH₃O—(CH₂CH₂O)₄₄—CH₂CH₂CH₂NH— | 50 |
| BT090 | CH₃O—(CH₂CH₂O)₄₄—CH₂CH₂NH— | 0 |
| BT070 | HO— | 50 |

As shown in Table B, derivatives of BT051 having one additional carbon in the linker (BT126) or one fewer carbon in the linker (BT123), and derivatives of BT070 having one additional carbon in the linker (BT125) or one fewer carbon in the linker (BT122) exhibit inhibition of neutrophil migration at a dose of 100 nM. For the purpose of Table B, the PEG-cyclosporine A conjugate/precursor is represented by the compound having the following formula:

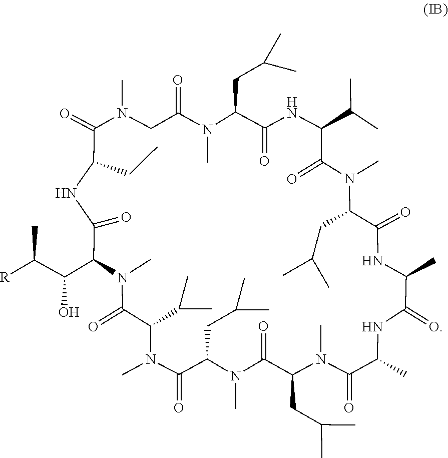

TABLE B
Inhibitory activity of PEG-cyclosporine A conjugates and precursors
in the neutrophil transmigration assay (compounds tested at 100 nM).

| Compound | R═ | % Inhibition |
|---|---|---|
| BT122 | COOH—CH₂CH₂CH₂CH₂— | 25 |
| BT123 | CH₃O—(CH₂CH₂O)₄₄—CH₂CH₂CH₂NH—CO—CH₂CH₂CH₂CH₂— | 50 |
| BT125 | COOH—CH₂CH₂CH₂CH₂CH₂CH₂— | 95 |
| BT126 | CH₃O—(CH₂CH₂O)₄₄—CH₂CH₂CH₂NH—CO—CH₂CH₂CH₂CH₂CH₂CH₂— | 50 |

These results demonstrate the surprising and unexpected capability of certain PEG-cyclosporine A conjugates/precursors to inhibit neutrophil migration. Accordingly, these results demonstrate that compounds of the current technology are useful in methods for inhibiting neutrophil migration, such as methods for the prevention or treatment of diseases or conditions caused by, resulting in, or otherwise associated with neutrophil migration.

### Example 3: Stability of PEG-Cyclosporine A Compounds in Simulated Intestinal Fluid, Simulated Gastric Fluid, and Feces

This example demonstrates that the PEG-Cyclosporine A compounds (including conjugates) of the present technology are stable in simulated intestinal fluid, simulated gastric fluid, and feces.

### A. BT090

Feces stability. Immediately prior to the study, male Sprague-Dawley rat feces were collected overnight over a mix of dry and wet ice. Feces were then homogenized in phosphate buffer at pH 6.5 and filtered to remove particles. BT090 (10 M) was incubated in duplicate at 37° C. for a total of 24 hours in the freshly prepared rat feces homogenate. At selected time points (0.5, 2, 6 and 24 hours), a 25 μL aliquot was taken from the incubation mixtures and quenched with 225 μL of acetonitrile containing the internal standards (IS). Samples were then centrifuged to pellet the precipitated proteins and the supernatants were diluted 5-fold. The 0 hour samples were prepared in pre-quenched feces homogenate towards the end of the incubation period. All quenched samples were then submitted to bioanalysis for LC-MS/MS quantification.

SGF FeSSIF Stability Procedure. BT090 (10 μM) was incubated in duplicate at 37° C. for a total of 6 hours in simulated gastric fluid (SGF) (Ricca, Arlington, TX) and Fed State Simulated Intestinal Fluid (FeSSIF) (Biorelevant, London, UK). The reactions were initiated by spiking the test articles (1 mM water stock solutions) in their respective buffers. At selected time points (0, 0.5, 1, 2, 4, and 6 hours), a 50 μL aliquot was taken from the incubation mixtures and frozen at −80° C. At the end of the incubation period, 200 μL of 50:50 acetonitrile:water containing the internal standards (IS) were added to every tube and vortexed thoroughly. The 0 hour samples were prepared in pre-quenched SGF or FeSSIF. All quenched samples were then submitted to bioanalysis for LC-MS/MS quantification.

Bioanalysis. BT090 samples were analyzed by LC-MS/MS using a Thermo Accela UPLC and a Thermo Q-Exactive mass spectrometer. The [M+H]+ adducts of the compounds and internal standards were monitored using positive mode electrospray ionization in exact mass mode. The analytes were injected onto a C₁₈ column and chromatographed using a reverse phase gradient with 0.1% formic acid in water and 0.1% formic acid in 20/80 isopropyl alcohol/acetonitrile mobile phases. Since there are several species in the polymeric mixture, the 5 most prominent peaks were integrated (masses tracked: 768.8, 779.8, 792.0, 801.8, 813.0; all 4+ charge state)

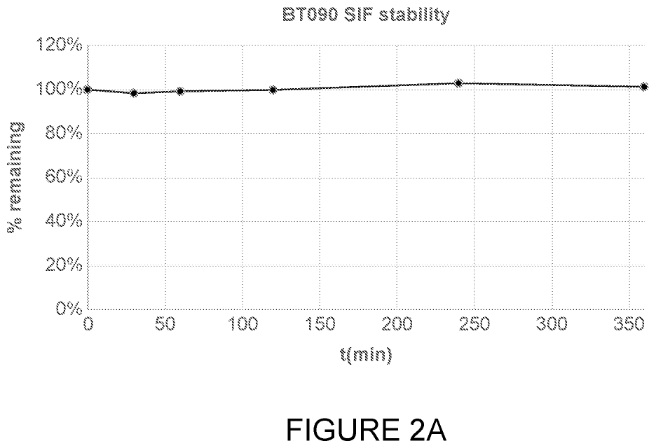

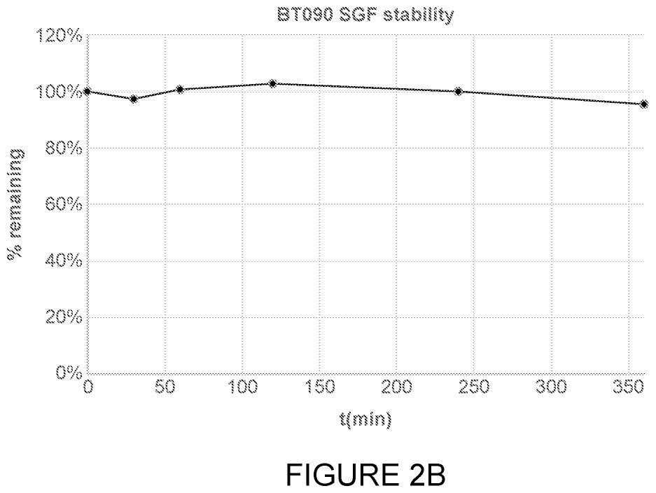

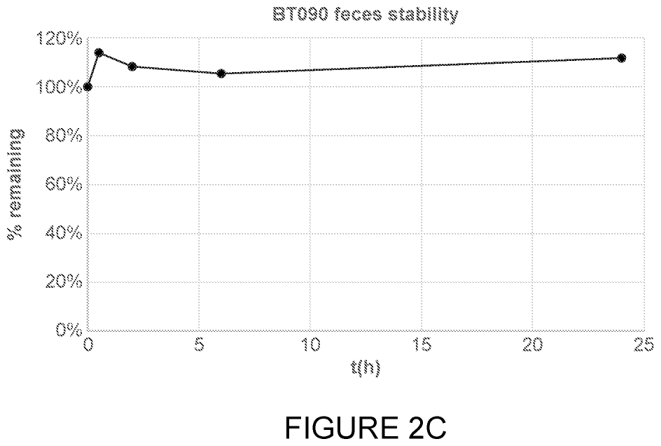

Results. As shown in FIG. 2A, BT090 is stable in simulated intestinal fluid. The compound was incubated for over 6 hours in FeSSIF and assayed for compound remaining at the time points indicated. As shown in FIG. 2B, BT090 is stable in simulated gastric fluid. The compound was incubated over 6 hours in SGF and assayed for compound remaining at the time points indicated. As shown in FIG. 2C, BT090 is stable in fresh rat fecal homogenate. The compound was incubated over 24 hours in feces and assayed for compound remaining at the time points indicated.

### B. BT051 and BT070

Feces stability. Immediately prior to the study, male Sprague-Dawley rat feces are collected overnight over a mix of dry and wet ice. Feces are then homogenized in phosphate buffer at pH 6.5 and filtered to remove particles. BT051 and BT070 (10 μM) are incubated in duplicate at 37° C. for a total of 24 hours in the freshly prepared rat feces homogenate. At selected time points (0.5, 2, 6 and 24 hours), a 25 μL aliquot is taken from the incubation mixtures and quenched with 225μL of acetonitrile containing the internal standards (IS). Samples are then centrifuged to pellet the precipitated proteins and the supernatants are diluted 5-fold. The 0 hour samples are prepared in pre-quenched feces homogenate towards the end of the incubation period. All quenched samples are then submitted to bioanalysis for LC-MS/MS quantification.

SGF FeSSIF Stability Procedure. BT051 and BT070 (10 μM) are incubated in duplicate at 37° C. for a total of 6 hours in simulated gastric fluid (SGF) (Ricca, Arlington, TX) and Fed State Simulated Intestinal Fluid (FeSSIF) (Biorelevant, London, UK). The reactions are initiated by spiking the test articles (1 mM water stock solutions) in their respective buffers. At selected time points (0, 0.5, 1, 2, 4, and 6 hours), a 50 μL aliquot is taken from the incubation mixtures and frozen at −80° C. At the end of the incubation period, 200 μL of 50:50 acetonitrile:water containing the internal standards (IS) are added to every tube and vortexed thoroughly. The 0 hour samples are prepared in pre-quenched SGF or FeSSIF. All quenched samples are then submitted to bioanalysis for LC-MS/MS quantification.

Bioanalysis. BT051 and BT070 samples are analyzed by LC-MS/MS using a Thermo Accela UPLC and a Thermo Q-Exactive mass spectrometer. The [M+H]+ adducts of the compounds and internal standards are monitored using positive mode electrospray ionization in exact mass mode. The analytes are injected onto a C₁₈ column and chromatographed using a reverse phase gradient with 0.1% formic acid in water and 0.1% formic acid in 20/80 isopropyl alcohol/acetonitrile mobile phases. Since there are several species in the polymeric mixture, the 5 most prominent peaks are integrated (masses tracked: 768.8, 779.8, 792.0, 801.8, 813.0; all 4+ charge state)

Results. It is predicted that BT051 and BT070 will be stable in simulated intestinal fluid, simulated gastric fluid, and fresh rat fecal homogenate.

These results show that compounds of the present technology are useful for methods comprising exposure of the compounds to intestinal fluid, gastric fluid, and fecal material.

### Example 4: Pharmacokinetics of PEG-Cyclosporine A Compounds of the Present Technology

This example demonstrates the pharmacokinetics of compounds of the present technology.

Three male, cannulated, fasted Sprague-Dawley rats were administered 10 mg/kg BT090, BT051, or BT070 orally at a dose rate of 10mL/kg at t-0 h. Blood samples were collected at the indicated time points and processed to plasma. Plasma concentrations of BT090 were assayed by LC-MS/MS. Feces were collected over t=0 to 4 h, 4-8 h, and 8-24 h, homogenized in buffer and assayed for BT090 concentrations by LC-MS/MS. Total feces weight collected for each time interval was recorded. BT090 was formulated in 1% NMP, 0.3% Tween-80 in 0.5% methylcellulose.

Tables 1-4 show, respectively, the oral bioavailability of compounds BT090, BT051, BT070 (the breakdown product of orally dosed BT051), and BT070 (dosed separately). As shown in Table 1, BT090 is not orally bioavailable. BT090, when dosed orally in rats at 10 mg/kg, was not detectable in the plasma from 15 minutes to 24 hours. Only rat “C” showed any detectable level at 15 min, and this level was close to the limit of detection of the assay (25 ng/mL). NQ-below the limit of detection of the bioanalytical assay. NC-not calculated due to lack of data. As shown in Table 2, BT051 is orally bioavailable. BT051, when dosed orally in rats at 10 mg/kg, was detectable in the plasma at 15 minutes in each of the rat subjects (A-E). As shown in Table 3, the breakdown product of BT051 (i.e., BT070) is not orally bioavailable following oral administration of BT051 in rats at 10 mg/kg. As shown in Table 4, BT070, when dosed separately at 10 mg/kg orally in rats, is present in the plasma in all subject rats from 15 minutes to 24 hours.

TABLE 1
BT090 Rat PK.

| | Animal ID | | | Mean | SD | |
|---|---|---|---|---|---|---|
| Time (h) | A | B | C | (ng/mL) | (ng/mL) | % CV |
| 0.25 | NQ | NQ | 27.0 | 27.0 | — | — |
| 0.5 | NQ | NQ | NQ | — | — | — |
| 1 | NQ | NQ | NQ | — | — | — |
| 2 | NQ | NQ | NQ | — | — | — |
| 4 | NQ | NQ | NQ | — | — | — |
| 6 | NQ | NQ | NQ | — | — | — |
| 8 | NQ | NQ | NQ | — | — | — |
| 24 | NQ | NQ | NQ | — | — | — |
| Cₘₐₓ (ng/mL) | NC | NC | 27.0 | 27.0 | — | — |
| tₘₐₓ (h) | NC | NC | 0.25 | 0.25 | — | — |
| AUCₗₐₛₜ (ng/mL*h) | NC | NC | NC | — | — | — |
| AUC_{inf} (ng/mL*h) | NC | NC | NC | — | — | — |
| % Extrapolation | NC | NC | NC | — | — | — |
| CL_F (mL/min/kg) | NC | NC | NC | — | — | — |
| M.R.T. po (h) | NC | NC | NC | — | — | — |

NQ: Not quantifiable.
No peak or below limit of quantification (LOQ = 25.0 ng/mL)
NC: Not calculated.

TABLE 2
BT051 Rat PK.

| | Animal ID | | | | | Mean | SD | |
|---|---|---|---|---|---|---|---|---|
| Time (h) | A | B | C | D | E | (ng/mL) | (ng/mL) | % CV |
| 0.25 | 22.5 | 35.2 | 35.7 | 42.5 | 23.1 | 31.8 | 8.7 | 27.4 |
| 0.5 | 28.3 | 56.2 | 38.1 | 14.4 | 26.6 | 32.7 | 15.6 | 47.6 |
| 1 | 16.0 | 51.5 | 28.4 | 13.3 | 18.9 | 25.6 | 15.5 | 60.6 |
| 2 | 11.1 | 34.1 | 12.8 | 10.7 | 11.3 | 16.0 | 10.2 | 63.5 |
| 4 | 9.2 | 17.3 | 10.2 | 24.5 | NQ | 15.3 | 7.1 | 46.4 |
| 6 | NQ | 7.7 | 8.7 | 21.6 | NQ | 12.7 | 7.7 | 60.9 |
| 8 | NQ | 7.0 | 7.4 | 8.4 | 62.7 | 21.4 | 27.5 | 128.8 |
| 24 | NQ | NQ | NQ | NQ | NQ | — | — | — |
| Cₘₐₓ (ng/mL) | 28.3 | 56.2 | 38.1 | 42.5 | 62.7 | 45.6 | 13.8 | 30.4 |
| tₘₐₓ (h) | 0.50 | 0.50 | 0.50 | 0.25 | 8.00 | 1.95 | 3.38 | 173.53 |
| AUCₗₐₛₜ (ng/mL*h) | 54.2 | 176.8 | 109.0 | 142.6 | 257.4 | 148.0 | 76.1 | 51.4 |
| AUC_{inf} (ng/mL*h) | 108.5* | 208.2 | 201.7* | 174.3 | 368.1* | 212.1 | 95.7 | 45.1 |
| % Extrapolation | 50.0 | 15.1 | 46.0 | 18.1 | 30.1 | 31.9 | 15.8 | 49.7 |
| CL_F (mL/min/kg) | 1535.8* | 800.5 | 826.4* | 956.4 | 452.8* | 914.4 | 394.2 | 43.1 |
| M.R.T. po (h) | 5.8* | 4.0 | 11.0* | 5.4 | 7.3* | 6.69 | 2.68 | 40.08 |

NQ: Not quantifiable.
No peak or below limit of quantification (LOQ = 5.0 ng/mL)
NC: Not calculated.

TABLE 3
BT070 (breakdown product of BT051) Rat PK.

| | Animal ID | | | | | Mean | SD | |
|---|---|---|---|---|---|---|---|---|
| Time (h) | A | B | C | D | E | (ng/mL) | (ng/mL) | % CV |
| 0.25 | NQ | NQ | NQ | NQ | NQ | — | — | — |
| 0.5 | 3.3 | NQ | NQ | NQ | NQ | 3.3 | — | — |
| 1 | NQ | NQ | NQ | NQ | NQ | — | — | — |
| 2 | NQ | NQ | NQ | NQ | NQ | — | — | — |
| 4 | NQ | NQ | NQ | NQ | NQ | — | — | — |
| 6 | NQ | NQ | NQ | NQ | NQ | — | — | — |
| 8 | NQ | NQ | NQ | NQ | NQ | — | — | — |
| 24 | NQ | NQ | NQ | NQ | NQ | — | — | — |
| Cₘₐₓ (ng/mL) | 3.3 | NC | NC | NC | NC | 3.3 | — | — |
| tₘₐₓ (h) | NC | NC | NC | NC | NC | — | — | — |
| AUCₗₐₛₜ (ng/mL*h) | NC | NC | NC | NC | NC | — | — | — |
| AUC_{inf} (ng/mL*h) | NC | NC | NC | NC | NC | — | — | — |
| % Extrapolation | NC | NC | NC | NC | NC | — | — | — |
| M.R.T. po (h) | NC | NC | NC | NC | NC | — | — | — |

NQ: Not quantifiable.
No peak or below limit of quantification (LOQ = 2.5 ng/mL)
NC: Not calculated.

TABLE 4
BT070 Rat PK.

| | Animal ID | | | Mean | SD | |
|---|---|---|---|---|---|---|
| Time (h) | A | B | C | (ng/mL) | (ng/mL) | % CV |
| 0.25 | 6273.9 | 1109.8 | 1684.1 | 3022.6 | 2830.3 | 93.6 |
| 0.5 | 7472.8 | 746.7 | 2546.3 | 3588.6 | 3482.1 | 97.0 |
| 1 | 4382.8 | 725.2 | 1796.5 | 2301.5 | 1880.4 | 81.7 |
| 2 | 2238.3 | 1114.4 | 1215.5 | 1522.8 | 621.7 | 40.8 |
| 4 | 1178.8 | 861.5 | 448.5 | 829.6 | 366.2 | 44.1 |
| 6 | 641.5 | 543.6 | 280.0 | 488.3 | 187.0 | 38.3 |
| 8 | 312.1 | 451.6 | 118.1 | 293.9 | 167.4 | 57.0 |
| 24 | 12.0 | 268.9 | 22.8 | 101.2 | 145.3 | 143.5 |
| Cₘₐₓ (ng/mL) | 7472.8 | 1114.4 | 2546.3 | 3711.2 | 3335.4 | 89.9 |
| tₘₐₓ (h) | 0.50 | 2.00 | 0.50 | 1.0 | 0.9 | 86.6 |
| AUCₗₐₛₜ (ng/mL*h) | 17560.9 | 11798.3 | 7249.5 | 12202.9 | 5167.6 | 42.3 |
| AUC_{inf} (ng/mL*h) | 17616.8 | 19158.4* | 7432.0 | 14735.7 | 6372.0 | 43.2 |
| % Extrapolation | 0.3 | 38.42 | 2.46 | 13.7 | 21.4 | 155.9 |
| CL_F (mL/min/kg) | 9.5 | 8.7* | 22.43 | 13.5 | 7.7 | 57.0 |
| M.R.T. po (h) | 3.1 | 25.0 | 4.2 | 10.8 | 12.3 | 114.4 |

NQ: Not quantifiable.
No peak or below limit of quantification (LOQ = 10 ng/mL)
NC: Not calculated.

Tables 5-8, show, respectively, the amount of BT090, BT051, BT070 (the breakdown product of BT051), and BT070 (dosed separately) present in feces of orally-dosed Sprague Dawley rats. Amount assumes that I gram of feces equals 1 ml.

TABLE 5
BT090 Amount Excreted Unchanged in Feces Following Oral Administration of a 10 mg/kg dose.

| | Animal A 255 g | | | Animal B 239 g | | | Animal C 283 g | | |
|---|---|---|---|---|---|---|---|---|---|
| | Conc.* | Feces | Amount^{‡} | Conc. | Feces | Amount | Conc. | Feces | Amount |
| Time | (μg/mL) | sample (g) | (μg) | (μg/mL) | sample (g) | (μg) | (μg/mL) | sample (g) | (μg) |
| 0-4 h | 0.84 | 0.82 | 0.68 | 1.44 | 0.11 | 0.16 | 1.48 | 0.50 | 0.7 |
| 4-8 h | 1109.2 | 1.10 | 1214.6 | 1132.6 | 0.56 | 632.8 | 1.88 | 1.53 | 2.9 |
| 8-24 h | 406.6 | 4.07 | 1653.5 | 1085.3 | 1.34 | 1458.4 | 443.0 | 5.24 | 2323.1 |
| Total (μg) | | | 2868.7 | | | 2091.4 | | | 2326.7 |
| % of dose | | | 112.5 | | | 87.5 | | | 82.2 |

*Concentration corrected for the dilution factor (10-fold for 0-4 h; 50-fold for 4-24 h)

TABLE 6
BT051 Amount Excreted Unchanged in Feces Following Oral Administration of a 10 mg/kg dose.

| | Animal A 237 g | | | Animal B 257 g | | | Animal C 243 g | | |
|---|---|---|---|---|---|---|---|---|---|
| Time | Conc.* | Feces | Amount^{‡} | Conc.* | Feces | Amount^{‡} | Conc.* | Feces | Amount^{‡} |
| Interval | (μg/mL) | sample (g) | (μg) | (μg/mL) | sample (g) | (μg) | (μg/mL) | sample (g) | (μg) |
| 0-4 h | NQ | 0.95 | NC | NC | NS | NC | NQ | 0.60 | NC |
| 4-8 h | 27.1 | 0.97 | 26.3 | NQ | 0.30 | NC | 102.5 | 0.73 | 74.8 |
| 8-24 h | 578.4 | 1.12 | 650.1 | 663.2 | 1.77 | 1175.6 | 321.6 | 3.38 | 1087.7 |
| 24-48 h | 199.3 | 4.69 | 934.4 | 223.1 | 3.83 | 853.7 | 5.8 | 6.40 | 37.0 |
| Total (μg) | | | 1610.7 | | | 2029.4 | | | 1199.6 |
| % of dose | | | 68.0 | | | 79.0 | | | 49.4 |

*Concentration corrected for the dilution factor.
NS: No sample
NC: Not calculated
NQ: Not quantifiable.
No peak or below limit of quantification (LOQ = 0.5 μg/mL)

TABLE 7
BT070 (breakdown product of BT051) Amount Excreted in Feces Unchanged Following Oral Administration of a 10 mg/kg dose of BT051.

| | Animal A 237 g | | | Animal B 257 g | | | Animal C 243 g | | | Animal D 240 g | | |
|---|---|---|---|---|---|---|---|---|---|---|---|---|
| | | Feces | | | Feces | | | Feces | | | Feces | |
| Time | Conc.* | sample | Amount^{‡} | Conc.* | sample | Amount^{‡} | Conc.* | sample | Amount^{‡} | Conc.* | sample | Amount^{‡} |
| Interval | (μg/mL) | (g) | (μg) | (μg/mL) | (g) | (μg) | (μg/mL) | (g) | (μg) | (μg/mL) | (g) | (μg) |
| 0-4 h | NQ | 0.95 | NC | NC | NS | NC | NQ | 0.60 | NC | NQ | 1.09 | NC |
| 4-8 h | NQ | 0.97 | NC | NQ | 0.30 | NC | NQ | 0.73 | NC | NS | NS | NC |
| 8-24 h | 12.7 | 1.12 | 14.3 | 6.0 | 1.77 | 10.6 | 10.3 | 3.38 | 34.8 | 8.7 | 3.58 | 31.2 |
| 24-48 h | 4.08 | 4.69 | 19.1 | 8.1 | 3.83 | 30.8 | 0.6 | 6.40 | 3.7 | 0.8 | 7.27 | 5.7 |
| Total (μg) | | | 33.4 | | | 41.4 | | | 38.5 | | | 36.9 |
| % Parent dose | | | 3.6 | | | 4.1 | | | 4.1 | | | 4.0 |

*Concentration corrected for the dilution factor.
NS: No sample
NC: Not calculated
NQ: Not quantifiable.
No peak or below limit of quantification (LOQ = 0.5 μg/mL)

TABLE 8
BT070 Amount Excreted Unchanged in Feces Following Oral Administration of a 10 mg/kg dose.

| | Animal A 272 g | | | Animal B 280 g | | | Animal C 262 g | | |
|---|---|---|---|---|---|---|---|---|---|
| Time | Conc.* | Feces | Amount^{‡} | Conc.* | Feces | Amount^{‡} | Conc.* | Feces | Amount^{‡} |
| Interval | (ng/mL) | sample (g) | (ng) | (ng/mL) | sample (g) | (ng) | (ng/mL) | sample (g) | (ng) |
| 0-4 h | NQ | 0.16 | — | 1417.4 | 0.40 | 561.4 | NQ | 1.63 | — |
| 4-8 h | 155322.5 | 1.17 | 181805.0 | NQ | 0.24 | — | 366163.0 | 2.39 | 875239.4 |
| 8-24 h | 333649.7 | 3.09 | 1030677.2 | 314317.5 | 0.80 | 252114.1 | 153005.0 | 6.64 | 1016595.6 |
| Total (ng) | | | 1212482.2 | | | 252675.5 | | | 1891835.1 |
| % of dose | | | 44.6 | | | 9.0 | | | 72.2 |

*Concentration corrected for the 1:9 dilution factor.
NQ: Not quantifiable.
No peak or below limit of quantification (LOQ = 1000 ng/mL)

### Example 5: Inhibition of FPR1 by Compounds of the Present Technology

This example demonstrates the use of compounds of the present technology for the inhibition of FPR1, as measured by the inhibition of fMLP-induced neutrophil migration.

### A. BT051

T84 colorectal cancer cells were grown to confluence on the bottom side of 12 well polycarbonate membrane inserts of a 24 well Transwell® plate. The confluent growth of these epithelial cells results in physiologically-relevant apical (luminal) and basolateral surfaces in the transwells. Freshly isolated and prepared human neutrophils were pretreated in compound (BT051 or cyclosporine A) for 1 hour on ice.

BT051 or cyclosporine A was added to the apical surface of the monolayer. The potent neutrophil chemoattractant, N-formyl-methionyl-leucyl-phenylalanine (fMLP) (100 nM), which activates neutrophil migration via its interaction with the FPR1 protein on neutrophils, was also added to the apical side of the monolayer. The compound-treated neutrophils were then added to the basolateral side and allowed to transmigrate through the monolayer for 2 hours. The number of neutrophils migrated to the apical chamber (due to the fMLP gradient) was then assayed by determination of myeloperoxidase activity (a neutrophil biomarker). Inhibition of transmigration is indicated by lower MPO activity in the apical chamber.

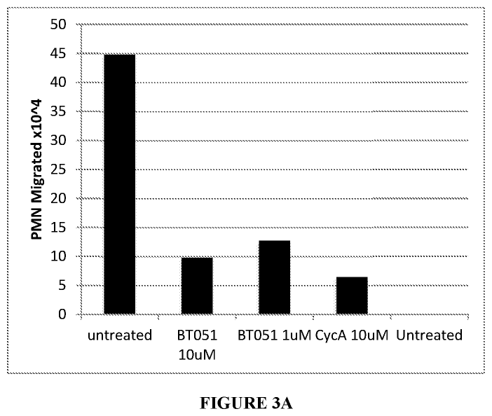

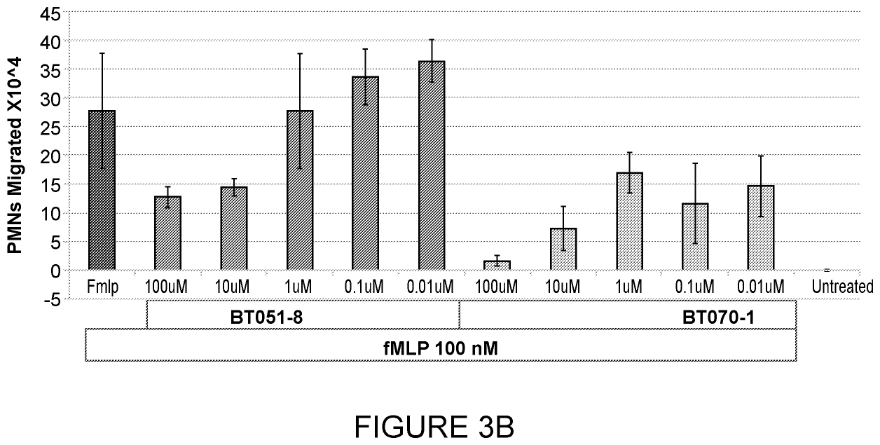

Results. As shown in FIG. 3A, BT051 inhibits fMLP-mediated polymorphonuclear cell (PMN) transmigration/activation. Human PMNs were pretreated with BT051 and added to the “basolateral” side of an epithelial monolayer in a transwell system. fMLP was present on the “apical” side of the transwell as a chemoattractant/neutrophil activator. BT051 blocked transmigration to the apical side and activation of PMNs significantly at 10 μM and 1 μM concentrations, similar to known FPR1 inhibitor cyclosporine A (CsA).

These results show that the compositions of the present technology inhibit FPR1. Accordingly, the compounds are useful in methods comprising the inhibition of FPR1, such as in the treatment of FPR1-mediated diseases and conditions, such as those associated with glucose intolerance.

### B. BT070 and BT090

T84 colorectal cancer cells are grown to confluence on the bottom side of 12 well polycarbonate membrane inserts of a 24 well Transwell® plate. The confluent growth of these epithelial cells results in physiologically-relevant apical (luminal) and basolateral surfaces in the transwells. Freshly isolated and prepared human neutrophils are pretreated in BT070, BT090, or cyclosporine A for 1 hour on ice.

BT070, BT090, or cyclosporine A is added to the apical surface of the monolayer. The potent neutrophil chemoattractant, N-formyl-methionyl-leucyl-phenylalanine (fMLP), which activates neutrophil migration, is also added to the apical side of the monolayer. The compound-treated neutrophils are then added to the basolateral side and allowed to transmigrate through the monolayer for 2 hours. The number of neutrophils migrated to the apical chamber (due to the fMLP gradient) is then assayed by determination of myeloperoxidase activity (a neutrophil biomarker). Inhibition of transmigration is indicated by lower MPO activity in the apical chamber.

Results. As shown in FIG. 3B, BT070 and BT051 inhibit fMLP-mediated polymorphonuclear cell (PMN) transmigration/activation. It is predicted that and BT090 will also inhibit fMLP-mediated PMN transmigration/activation. BT070 inhibited PMN transmigration/activation by approximately 50%. In this experiment, BT051 inhibited PMN transmigration/activation by 50% at a 10 μM concentration.

These results show (or will show) that the compositions of the present technology inhibit FPR1. Accordingly, the compounds are useful in methods comprising the inhibition of FPR1, such as in the treatment of FPR1-mediated diseases and conditions, such as those associated with glucose intolerance.

### Example 6: Compounds of the Present Disclosure Improve Glucose Tolerance in Diet-Induced Obese Mice

This example will demonstrate that compounds of the present disclosure modulate GLP-1 levels and glucose tolerance in a mouse model of obesity.

It has previously been shown that 1) fMLF is increased in HFD/obese mice and requires Fprl to induce chemotaxis, 2) the loss of Fprl in HFD/obese mice improves glucose tolerance and is GLP-1 dependent, 3) GLP-1 secretion is dependent on Fprl expression, and 4) pharmacological control of Fprl activity using the antagonist cFLFLF modulates GLP-1 levels and glucose tolerance.

The effects of compounds of the present disclosure on glucose metabolism in vivo are assessed using methods known in the art and briefly described below.

Methods: Frp1 knockout (Frp1-KO) and Glplr-KO mice are generated as described in Wollam et al. (Diabetes 68:1415-1426 (2019)). Only male mice are used in this study, fed ad libitum and maintained on a 12-h light/dark cycle. At 8 weeks of age, mice are placed on a 60% HFD (Research Diets) or normal chow diet (NCD) (13.5% fat; LabDiet) for 8-10 weeks before analyses. For broad-spectrum antibiotic treatment, mice are subject to daily oral gavage (OG) for 2 weeks with water alone or containing a mixture of antibiotics (Sigma-Aldrich): 0.5 mg/mL vancomycin HCl, 1 mg/mL ampicillin sodium salt, 1 mg/mL neomycin sulfate, 1 mg/mL metronidazole, and 1 mg/mL gentamycin sulfate. Mice are treated with the GlpIr antagonist exendin 9-39 (Sigma-Aldrich) or saline vehicle in a 0.2 mg/kg dose 20 min before metabolic studies. fMLF (Sigma-Aldrich) is administered at 1 mg/kg in saline via OG 1 h prior to analyses. Compounds of the present disclosure are provided daily at 0.5 mg/kg by OG for 2 weeks prior to analyses. For all drug treatments, body weight and food intake are monitored prior to and during the treatment period. GF C₅₇BL/6 mice are maintained in a standard animal facility. GF mice are fed either an autoclaved chow diet (2019S Teklad Global 19% Protein Extruded Rodent Diet; Harlan Laboratories) or an irradiated sterilized 60% HFD (06414 Teklad), as previously described. For fecal transplantation, 8- to 10-week-old GF mice are gavaged with fecal suspensions from either WT HFD or Fpr1-KO HFD animals (50 mg/mL). Animal housing and procedures are performed according to appropriate Institutional Animal Care and Use Committee-approved protocols.

Further methods include the use of animal models for disease conditions caused by or associated with defects in glucose tolerance, plasma insulin levels, and GLP-1 levels, such as those listed in Table C below.

TABLE C

| Disease | Animal Model |
|---|---|
| Diabetes | KK-A^{Y} mice are a diabetes model with a less severe obesity |
| | phenotype than other models. Other models for various types of |
| | diabetes are also known in the art and can be employed in the |
| | methods described herein. |
| Obesity | High-fat-diet induced obese mouse are a popular model that mimics |
| | human obesity. |
| Klinefelter Syndrome | 41, XXY* mice are a commonly used model. |
| Metabolic Syndrome | There are several mouse models for MetS, several of which are |
| | described in e.g., Kennedy et al, Dis. Model Mech. 3(304): 156-166 |
| | (2010). |
| Turner Syndrome | The XO mouse is the standard mouse model for turner syndrome. |
| Wolfram Syndrome | WFS1-Rats are an accepted model. |
| Cystic Fibrosis | —/—CFTR Pigs are the best model for Cystic Fibrosis associated |
| | Diabetes in adult humans, but a —/—CFTR ferret model also exists that |
| | may be easier to use in lab. |
| Friedrich's Ataxia | The pancreas specific frataxin knockout mouse model would be the |
| | best FA model for our purposes. |
| Multiple Sclerosis | The EAE (experimental autoimmune/allergic encephalomyelitis) |
| | mouse model of MS is the best model for understanding the |
| | inflammatory aspects of the disease. |
| Down Syndrome | Ts65Dn mice are a common model for down syndrome. |
| Muscular Dystrophy | mdx Mice are a commonly used model for Duchenne Muscular |
| | Dystrophy. |
| Polycystic Ovary | PCOS can be modeled in mice with daily dihydrotestosterone (DHT) |
| Syndrome | treatment. |
| Chronic Renal | Subtotal nephrectomy can be used to induce chronic renal failure in |
| Failure | nice. |
| HIV infection | No perfect models exist for HAART therapy, but there are two |
| paired with | options. HAART treatment can be modeled by drug treatment in |
| HAART | healthy mice or there is a HIV-1 transgenic rat model. |

Metabolic Studies: Mice are fasted for 6 h prior to conducting insulin tolerance tests and glucose tolerance tests (GTTs), which have been described previously.

GLP-I Detection: Blood serum is collected from mice fasted for 6 h, immediately before (basal) and 10-15 min after dextrose gavage, in the presence of DPP-4 (dipeptidyl peptidase 4) inhibitor (EMD Millipore). Postprandial blood collection is performed 10 min after gavage in NCD mice and 15 min after gavage in HFD mice. Analysis of GLP-1 is conducted by High Sensitivity GLP-1 Active ELISA Kit, Chemiluminescent (Millipore Sigma).

Glucose-Stimulated Insulin Secretion: Primary murine islets are isolated as previously described . Secreted insulin is normalized to the corresponding intracellular insulin levels in the cell pellet of each sample.

Glucose-Stimulated GLP-1 Secretion Assay: mGLUTag cells are stimulated with 3 mmol/L glucose in the presence or absence of Fprl agonists (fMLF) for 4 h.

Plasma Protein Measurements: Fasting insulin, glucagon, total GLP-1, and active GLP-1 levels are measured by ELISA (Alpco, Mercodia, and EMD Millipore, respectively). Other fasting plasma proteins are measured by MILLIPLEX Multiplex Assay (EMD Millipore).

Results: It is predicted that the mice will show increased glucose tolerance, increased insulin sensitivity, and/or increased GLP-1 levels, without changing body weight or food intake. It is further predicted that the improved glucose tolerance will be dependent on the Glp1r, and that treatment will have no effect in HFD Glp1r-KO littermates.

These results will show that compounds of the present disclosure are effective for increasing glucose tolerance, plasma insulin levels, and GLP-1 levels in subjects in need thereof. Accordingly, compounds of the present disclosure are useful in the treatment of disease conditions caused by or associated with defects in glucose tolerance, plasma insulin levels, and GLP-1 levels.

### Example 7: Compounds of the Present Disclosure Modulate GLP-1 Levels and Glucose Tolerance in Human Subjects

This example will demonstrate that compounds of the present disclosure modulate GLP-1 levels and glucose tolerance in human subjects.

Human subjects having a disease or condition caused by or associated with defects in glucose tolerance, plasma insulin levels, and GLP-1 levels are recruited accordingly to clinically appropriate criteria. Relevant disease indications include, but are not limited to type 2 diabetes mellitus, type 1 diabetes mellitus, gestational diabetes mellitus, neonatal diabetes, maturity-onset diabetes of the young, drug-induced diabetes, chemical-induced diabetes, hyperglycemia, impaired glucose tolerance (IGT), impaired fasting glucose (IFT), obesity, insulin resistance, metabolic syndrome, asthma, chronic obstructive pulmonary disease (COPD), Alzheimer's disease, cancers, Klinefelter Syndrome, Turner Syndrome, Wolfram Syndrome, Cystic Fibrosis, Friedreich's ataxia, multiple sclerosis, down syndrome, muscular dystrophy, polycystic ovary syndrome (PCOS), uremia, cirrhosis, chronic renal failure, and HAART-treated HIV infection.

Subjects are administered compounds of the present disclosure according to clinically acceptable protocols.

In some embodiments, subjects are administered the compounds once, twice, three times, or more per day. In some embodiments, subjects are administered the compounds once, twice, three times, or more per week. In some embodiments, subjects are administered the compounds once, twice, three times, or more per month.

In some embodiments, subjects are administered the compounds for a period or one, two, three, or more months. In some embodiments, subjects are administered the compounds for a period of up to 12 months.

Glucose tolerance, plasma insulin levels, and GLP-1 levels are measured using methods known in the art.

Results: It is predicted that the subjects will show increased glucose tolerance, increased insulin sensitivity, and/or increased GLP-1 levels, without changing body weight or food intake. It is further predicted that the improved glucose tolerance will be dependent on the Glp1r.

These results will show that compounds of the present disclosure are effective for increasing glucose tolerance, plasma insulin levels, and GLP-1 levels in subjects in need thereof. Accordingly, compounds of the present disclosure are useful in the treatment of disease conditions caused by or associated with defects in glucose tolerance, plasma insulin levels, and GLP-1 levels.

### Example 8: Compounds of the Present Disclosure Improve Glucose Tolerance and Insulin Sensitivity in Diet-Induced Obese Mice

This example demonstrates that compounds of the present disclosure improve glucose tolerance and insulin sensitivity in diet-induced obese (DIO) mouse model.

Methods: All procedures were performed in accordance with the Guide for the Care and Use of Laboratory Animals (revised 1996) and French laws. Male C₅₇BL6/J mice (Jackson Laboratories USA) mice were housed in standard conditions with whole phase high-fat diet (Research Diets, D12492) and tap water provided ad libitum to generate diet induced obesity (DIO). A total of 24 mice were housed as such in order to obtain 20 evaluable animals at the end of the experimental phase. Mice were acclimated in said conditions for two weeks prior to treatment.

After the acclimation period, the mice were weighed, fasted for 6 hours, and assayed for blood (25μL/EDTA) glucose and plasma levels using a glucometer and a commercial mouse insulin ELISA assay. Fasting glucose and plasma levels were used to calculate the HOMA-IR (Homeostasis Model Assessment of Insulin Resistance=(fasting insulin (μU/mL)×fasting glucose (mmol/L))/22.5). The HOMA-IR index is a widely used tool for estimating insulin resistance. The mice were then randomized into two groups (n=10 mice/group) according to their (1) HOMA-IR and (2) body weight, with 4 mice excluded for extreme HOMA-IR and body weight values.

The control group received vehicle (PBS) orally once a day at a dose volume of 10 mL/kg. The treatment group received BT051 in vehicle (PBS) orally once a day at a dosage of 200 mg/kg and a volume of 10 mL/kg. The dosage regimen is shown in Table 9.

| Group | #mice/ | | | | Dose | Treatment |
|---|---|---|---|---|---|---|
| No | group | Treatment | Route | Dose | volume | duration |
| 1 | 10 | Vehicle | p.o., | — | 10 | 2 |
| | | | QD | | mL/kg | weeks |
| 2 | 10 | BT051 | p.o., | 200 | 10 | 2 |
| | | | QD | mg/kg | mL/kg | weeks |

Mice were weighed at weekly intervals during treatment. At 2 weeks of treatment, mice were weighed, treated a final time, and fasted for 6 hours. Then, an oral glucose tolerance test (OGTT) was performed using a glucose load of 1g/kg body weight and blood glucose measurements were taken at −30, 0, 15, 30, 60, 90, and 120 minutes from glucose load. At −30 and 15 minutes from glucose load, blood (15μL/EDTA) was collected for plasma insulin measurements. The ratio of plasma insulin levels at 15 minutes post-glucose load to 30 minutes pre-glucose load was calculated (T15/-30) to determine if there was a relative change in plasma insulin.

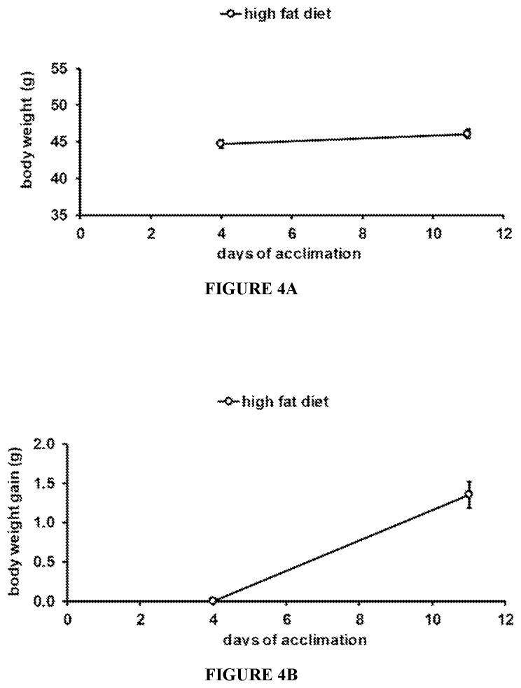

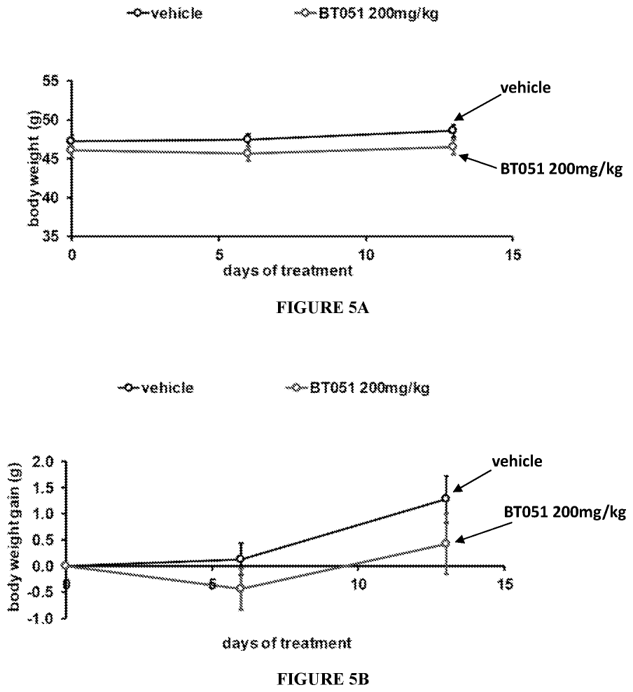

Results: During the acclimation period, the DIO mice gained weight as expected (FIGS. 4A-4B). During the treatment period, body weight was recorded every week (FIG. 5A). During the first week, the BT051-treated group tended to lose weight and gained weight again the second week (FIG. 5B). See also Tables 10 and 11 below.

TABLE 10
Body weight and body weight gain during acclimation period.

| | | body weight (g)/days | | body weight gain (g)/ | |
|---|---|---|---|---|---|
| | | of acclimation | | days of acclimation | |
| diet | mouse # | 4 | 11 | 4 | 11 |
| high | 1 | 48.5 | 48.6 | 0.0 | 0.1 |
| fat | 2 | 45.5 | 47.8 | 0.0 | 2.3 |
| diet | 3 | 46.9 | 49.3 | 0.0 | 2.4 |
| | 4 | 46.2 | 47.3 | 0.0 | 2.1 |
| | 5 | 47.4 | 47.6 | 0.0 | 0.2 |
| | 6 | 46.7 | 48.2 | 0.0 | 1.5 |
| | 7 | 46.3 | 46.4 | 0.0 | 0.1 |
| | 8 | 42.6 | 42.0 | 0.0 | −0.6 |
| | 9 | 49.7 | 51.0 | 0.0 | 1.3 |
| | 10 | 42.0 | 42.9 | 0.0 | 0.9 |
| | 11 | 40.9 | 44.0 | 0.0 | 3.1 |
| | 12 | 41.7 | 42.7 | 0.0 | 1.0 |
| | 13 | 45.9 | 48.4 | 0.0 | 2.5 |
| | 14 | 43.4 | 44.8 | 0.0 | 1.4 |
| | 15 | 44.4 | 47.0 | 0.0 | 2.6 |
| | 16 | 49.6 | 61.9 | 0.0 | 2.3 |
| | 17 | 46.7 | 47.1 | 0.0 | 0.4 |
| | 18 | 48.0 | 49.9 | 0.0 | 1.9 |
| | 19 | 47.3 | 49.9 | 0.0 | 2.6 |
| | 20 | 41.8 | 41.4 | 0.0 | −0.4 |
| | 21 | 45.9 | 47.9 | 0.0 | 2.0 |
| | 22 | 38.9 | 40.0 | 0.0 | 1.1 |
| | 23 | 43.6 | 45.3 | 0.0 | 1.7 |
| | 24 | 47.4 | 49.3 | 0.0 | 1.9 |
| | 25 | 52.3 | 54.2 | 0.0 | 1.9 |
| | 26 | 43.5 | 44.2 | 0.0 | 0.7 |
| | 27 | 41.6 | 44.1 | 0.0 | 2.5 |
| | 28 | 40.3 | 42.3 | 0.0 | 2.0 |
| | 29 | 40.5 | 41.0 | 0.0 | 0.5 |
| | 30 | 44.1 | 46.2 | 0.0 | 2.1 |
| | 31 | 46.3 | 47.8 | 0.0 | 1.5 |
| | 32 | 44.3 | 45.0 | 0.0 | 0.7 |
| | 33 | 46.3 | 47.0 | 0.0 | 0.7 |
| | 34 | 36.1 | 35.4 | 0.0 | −0.7 |
| | 35 | 48.1 | 48.5 | 0.0 | 0.4 |
| | 36 | 40.5 | 42.5 | 0.0 | 2.0 |
| | mean | 44.7 | 46.1 | 0.0 | 1.4 |
| | sd | 3.4 | 3.7 | 0.0 | 1.0 |
| | sem | 0.6 | 0.6 | 0.0 | 0.2 |
| | n | 36 | 36 | 36 | 36 |

TABLE 11
Body weight and body weight gain during
treatment period with vehicle or BT051.

| | | body weight (g)/ | | | body weight gain (g)/ | | |
|---|---|---|---|---|---|---|---|
| | | days of treatment | | | days of treatment | | |
| treatment | mouse # | 0 | 6 | 13 | 0 | 6 | 13 |
| vehicle | 1 | 48.6 | 47.0 | 47.7 | 0.0 | −1.6 | −0.9 |
| | 2 | 47.8 | 48.1 | 48.1 | 0.0 | 0.3 | 0.3 |
| | 3 | 49.3 | 48.6 | 49.8 | 0.0 | −0.7 | 0.5 |
| | 4 | 47.3 | 46.6 | 47.1 | 0.0 | −0.7 | −0.2 |
| | 9 | 51.0 | 51.7 | 53.2 | 0.0 | 0.7 | 2.2 |
| | 10 | 42.9 | 43.7 | 45.0 | 0.0 | 0.8 | 2.1 |
| | 11 | 44.0 | 45.3 | 46.9 | 0.0 | 1.3 | 2.9 |
| | 19 | 49.9 | 49.8 | 51.2 | 0.0 | −0.1 | 1.3 |
| | 32 | 45.0 | 44.8 | 46.1 | 0.0 | −0.2 | 1.1 |
| | 33 | 47.0 | 48.5 | 50.5 | 0.0 | 1.5 | 3.5 |
| | mean | 47.3 | 47.4 | 48.6 | 0.0 | 0.1 | 1.3 |
| | sd | 2.6 | 2.4 | 2.5 | 0.0 | 1.0 | 1.4 |
| | sem | 0.8 | 0.8 | 0.8 | 0.0 | 0.3 | 0.4 |
| | n | 10 | 10 | 10 | 10 | 10 | 10 |
| BT051 | 12 | 42.7 | 42.0 | 44.0 | 0.0 | −0.7 | 1.3 |
| 200 mg/kg | 13 | 48.4 | 47.8 | 49.7 | 0.0 | −0.6 | 1.3 |
| | 14 | 44.8 | 44.7 | 45.9 | 0.0 | −0.1 | 1.1 |
| | 16 | 51.9 | 52.2 | 53.3 | 0.0 | 0.3 | 1.4 |
| | 17 | 47.1 | 46.1 | 47.2 | 0.0 | −1.0 | 0.1 |
| | 21 | 47.9 | 47.3 | 45.4 | 0.0 | −0.6 | −2.5 |
| | 23 | 45.3 | 45.3 | 45.8 | 0.0 | 0.0 | 0.5 |
| | 27 | 44.1 | 45.2 | 45.7 | 0.0 | 1.1 | 1.6 |
| | 28 | 42.3 | 42.9 | 44.9 | 0.0 | 0.6 | 2.6 |
| | 30 | 46.2 | 42.8 | 43.1 | 0.0 | −3.4 | −3.1 |
| | mean | 46.1 | 45.6 | 46.5 | 0.0 | −0.4 | 0.4 |
| | sd | 2.9 | 3.0 | 3.0 | 0.0 | 1.2 | 1.8 |
| | sem | 0.9 | 0.9 | 0.9 | 0.0 | 0.4 | 0.6 |
| | n | 10 | 10 | 10 | 10 | 10 | 10 |

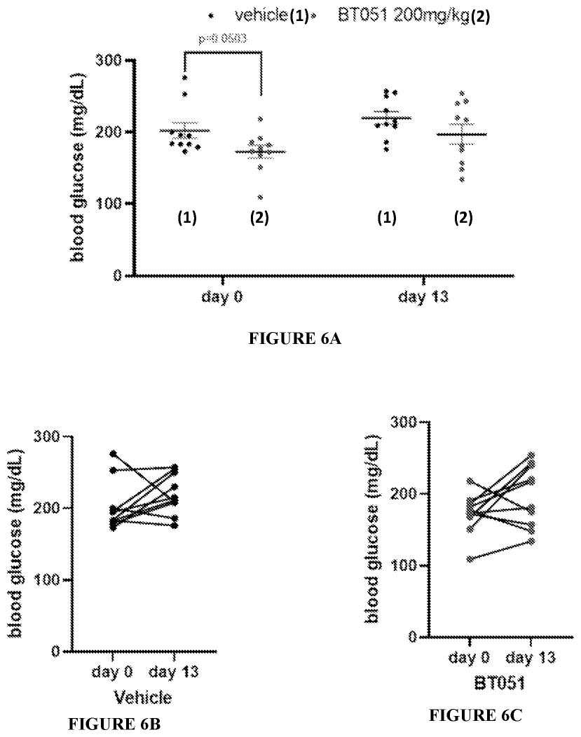

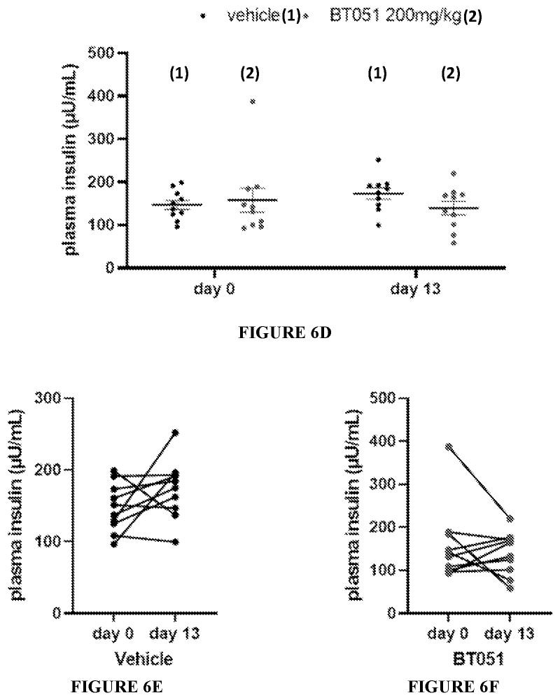

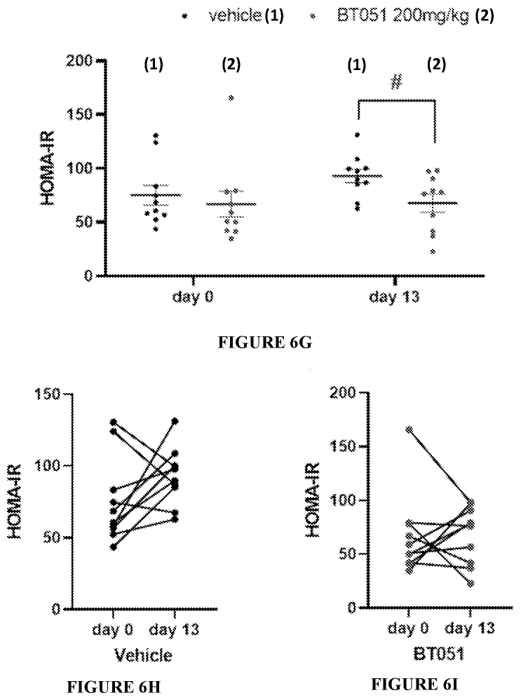

After two weeks of treatment, the BT051-treated mice had lower levels of fasting blood glucose (vehicle: 220 mg/dL versus BT051: 197 mg/dL) (FIGS. 6A-6C) and plasma insulin levels (vehicle: 173μU/mL versus BT051: 139μU/mL) (FIGS. 6D-6F) as compared to the control (vehicle-treated) mice, leading to a significant decrease of the HOMA-IR index (FIGS. 6G-6I). Thus, while fasting glucose levels between day 0 and day 13 increased to approximately the same extent in the vehicle-treated group and the BT051-treated group (FIGS. 6A-6C), the plasma insulin levels increased in the vehicle-treated group (day 0: 147.1 μU/mL versus day 13: 173.7 μU/mL), but decreased in the BT051-treated group (day 0: 158 μU/mL versus day 13: 139.5 μU/mL) (FIGS. 6D-6F). See also Table 12 below.

TABLE 12
Blood glucose, plasma insulin, and HOMA-IR before and
after 2 week treatment period with vehicle or BT051.

| | | blood glucose | | plasma insulin | | | |
|---|---|---|---|---|---|---|---|
| | | (mg/dL) | | (μU/mL) | | HOMA-IR | |
| treatment | mouse # | day 0 | day 13 | day 0 | day 13 | day 0 | day 13 |
| vehicle | 1 | 276 | 209 | 191.4 | 192.9 | 130.5 | 99.6 |
| | 2 | 195 | 215 | 173.4 | 184.3 | 83.3 | 97.8 |
| | 3 | 179 | 208 | 137.2 | 174.9 | 60.7 | 89.8 |
| | 4 | 253 | 257 | 198.9 | 137.0 | 124.0 | 86.9 |
| | 9 | 183 | 211 | 129.0 | 252.0 | 58.3 | 131.3 |
| | 10 | 200 | 188 | 151.2 | 146.8 | 74.7 | 67.4 |
| | 11 | 196 | 255 | 108.4 | 99.7 | 52.4 | 62.8 |
| | 19 | 184 | 250 | 125.0 | 162.3 | 56.8 | 100.2 |
| | 32 | 173 | 230 | 160.6 | 191.4 | 68.6 | 108.7 |
| | 33 | 183 | 176 | 96.4 | 196.1 | 43.6 | 85.2 |
| | mean | 202 | 220 | 147.1 | 173.7 | 75.3 | 93.0 |
| | sd | 34 | 28 | 34.2 | 41.0 | 29.6 | 19.7 |
| | sem | 11 | 9 | 10.8 | 13.0 | 9.4 | 6.2 |
| | n | 10 | 10 | 10 | 10 | 10 | 10 |
| BT051 | 12 | 168 | 243 | 100.3 | 132.2 | 41.6 | 79.3 |
| 200 mg/kg | 13 | 186 | 254 | 109.0 | 124.5 | 50.0 | 78.1 |
| | 14 | 151 | 240 | 93.1 | 164.3 | 34.7 | 97.4 |
| | 16 | 173 | 181 | 387.3 | 219.8 | 185.5 | 98.0 |
| | 17 | 218 | 175 | 147.3 | 176.6 | 79.3 | 76.3 |
| | 21 | 172 | 157 | 184.5 | 58.7 | 78.3 | 22.8 |
| | 23 | 191 | 220 | 142.0 | 76.8 | 66.9 | 41.7 |
| | 27 | 182 | 217 | 131.3 | 169.0 | 59.0 | 90.6 |
| | 28 | 109 | 134 | 189.1 | 171.2 | 50.9 | 56.6 |
| | 30 | 177 | 148 | 96.4 | 101.6 | 42.1 | 37.1 |
| | mean | 173 | 197 | 158.0 | 139.5 | 66.8 | 67.8 |
| | sd | 28 | 43 | 87.6 | 50.0 | 37.8 | 26.6 |
| | sem | 9 | 14 | 27.7 | 15.8 | 12.0 | 8.4 |
| | n | 10 | 10 | 10 | 10 | 10 | 10 |

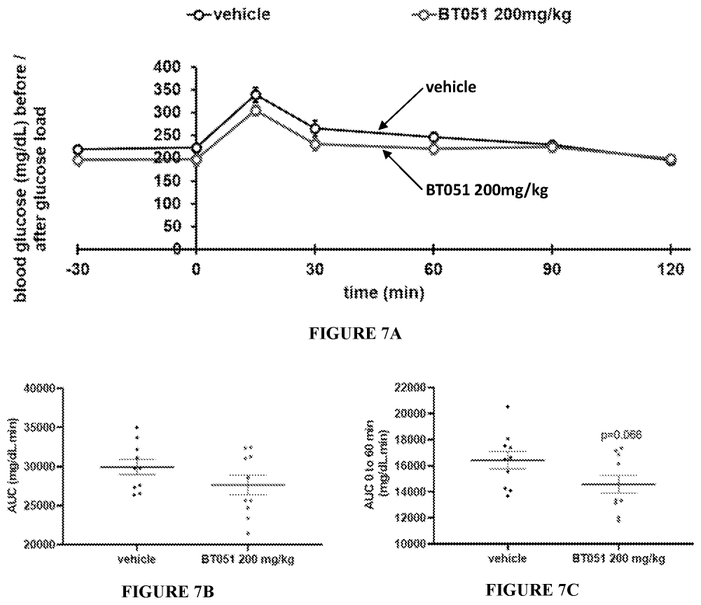

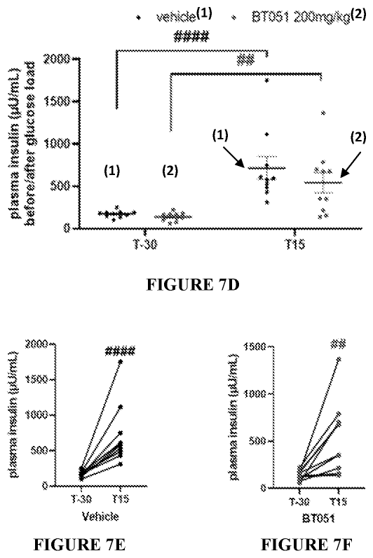

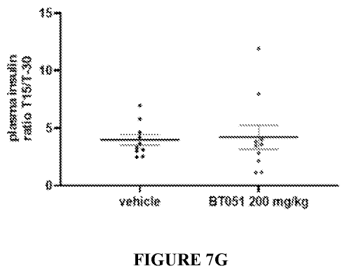

During oral glucose tolerance testing (OGTT), the blood glucose levels of BT051-treated mice were lower than in vehicle-treated mice from −30 minutes to 60 minutes from oral glucose load (FIG. 7A). Area under curve (AUC) analysis demonstrated a general decrease in the BT051-treated mice blood glucose AUC as compared to vehicle-treated mice, with a more pronounced difference between the BT051-treated and vehicle-treated control mice blood glucose AUC in the 0 to 60 minutes from treatment period (FIGS. 7B-7C). The T15/-30 ratio did not show an accompanying post-glucose load increase in blood plasma levels in the treatment group relative to the control group (FIGS. 7D-7G), indicating that the observed shifts in blood glucose levels are due to improved insulin sensitivity and not simply an increase in insulin. See also Table 13 below.

TABLE 13
Oral glucose tolerance test at the end of the treatment period with vehicle or BT051.

| | | | | | | | | | | AUC | | | plasma |
|---|---|---|---|---|---|---|---|---|---|---|---|---|---|
| | | blood glucose (mg/dL) before/ | | | | | | | AUC | (mg/dL · | plasma insulin | | insulin |
| | | after glucose load | | | | | | | (mg/dL · | min) 0 to | (μU/mL) | | ratio |
| treatment | mouse # | −30 | 0 | 15 | 30 | 60 | 90 | 120 | min) | 60 min | T-30 | T15 | T15/T-30 |
| vehicle | 1 | 209 | 196 | 325 | 203 | 211 | 212 | 194 | 28528 | 14083 | 193 | 1118 | 5.79 |
| | 2 | 215 | 240 | 299 | 255 | 235 | 195 | 177 | 27578 | 15548 | 184 | 610 | 3.31 |
| | 3 | 208 | 231 | 354 | 244 | 215 | 205 | 183 | 36378 | 14258 | 175 | 433 | 2.48 |
| | 4 | 257 | 227 | 304 | 183 | 221 | 243 | 203 | 27345 | 13595 | 137 | 577 | 4.21 |
| | 9 | 211 | 212 | 362 | 274 | 229 | 226 | 195 | 29760 | 18520 | 252 | 1754 | 6.96 |
| | 10 | 185 | 211 | 384 | 264 | 283 | 286 | 221 | 33858 | 17528 | 147 | 531 | 3.52 |
| | 11 | 255 | 225 | 327 | 264 | 285 | 247 | 217 | 31148 | 16508 | 160 | 310 | 3.11 |
| | 19 | 250 | 225 | 390 | 257 | 272 | 256 | 203 | 32205 | 17400 | 162 | 760 | 4.62 |
| | 32 | 230 | 272 | 425 | 334 | 306 | 234 | 192 | 35010 | 20520 | 191 | 485 | 2.54 |
| | 33 | 178 | 198 | 321 | 374 | 224 | 191 | 175 | 29790 | 18075 | 196 | 594 | 3.03 |
| | mean | 220 | 224 | 339 | 265 | 246 | 230 | 196 | 29941 | 16424 | 174 | 716 | 3.97 |
| | sd | 28 | 22 | 51 | 56 | 33 | 30 | 15 | 3035 | 2110 | 41 | 424 | 1.46 |
| | sem | 9 | 7 | 16 | 18 | 10 | 9 | 5 | 960 | 667 | 13 | 134 | 0.46 |
| | n | 10 | 10 | 10 | 10 | 10 | 10 | 10 | 10 | 10 | 10 | 10 | 10 |
| BT051 | 12 | 243 | 222 | 358 | 287 | 244 | 268 | 235 | 32378 | 17153 | 132 | 156 | 1.18 |
| 200 mg/kg | 13 | 254 | 215 | 382 | 247 | 237 | 277 | 218 | 31268 | 18155 | 125 | 139 | 1.12 |
| | 14 | 240 | 214 | 304 | 221 | 230 | 247 | 210 | 28588 | 14588 | 164 | 364 | 2.16 |
| | 16 | 181 | 198 | 263 | 209 | 213 | 209 | 186 | 25613 | 13328 | 220 | 788 | 3.58 |
| | 17 | 175 | 215 | 277 | 211 | 162 | 209 | 204 | 25605 | 13385 | 177 | 674 | 3.81 |
| | 21 | 157 | 127 | 280 | 183 | 167 | 158 | 181 | 21461 | 11771 | 59 | 700 | 11.33 |
| | 23 | 220 | 200 | 321 | 292 | 298 | 248 | 228 | 32415 | 17355 | 77 | 216 | 2.81 |
| | 27 | 217 | 273 | 314 | 282 | 251 | 245 | 201 | 31820 | 16880 | 159 | 677 | 4.01 |
| | 28 | 134 | 143 | 384 | 173 | 188 | 195 | 178 | 23385 | 12045 | 171 | 1386 | 7.98 |
| | 30 | 148 | 168 | 287 | 209 | 192 | 205 | 169 | 24713 | 13148 | 102 | 352 | 3.48 |
| | mean | 197 | 197 | 305 | 231 | 221 | 226 | 199 | 27645 | 14580 | 139 | 542 | 4.20 |
| | sd | 43 | 42 | 34 | 43 | 39 | 38 | 24 | 3957 | 2143 | 50 | 349 | 3.33 |
| | sem | 14 | 13 | 11 | 14 | 12 | 11 | 8 | 1264 | 678 | 16 | 120 | 1.05 |
| | n | 10 | 10 | 10 | 10 | 10 | 10 | 10 | 10 | 10 | 10 | 10 | 10 |

These results demonstrate that the compounds of the present disclosure are effective for increasing glucose tolerance and insulin sensitivity in a widely accepted, mammalian model of obesity and glucose intolerance that mimics obesity in humans. Accordingly, these results demonstrate the usefulness of compounds of the present disclosure in methods for the treatment or prevention of diseases or conditions that are caused by or associated with glucose intolerance.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

Each and every publication and patent mentioned in the above specification is herein incorporated by reference in its entirety for all purposes. Various modifications and variations of the described methods and system of the present technology will be apparent to those skilled in the art without departing from the scope and spirit of the present technology. Although the present technology has been described in connection with specific embodiments, the present technology as claimed should not be unduly limited to such specific embodiments. Indeed, various modifications of the described modes for carrying out the present technology which are obvious to those skilled in the art and in fields related thereto are intended to be within the scope of the following claims.

1. A method for treating glucose intolerance in a mammalian subject in need thereof, comprising administering to the subject a therapeutically effective amount of a compound of formula I: 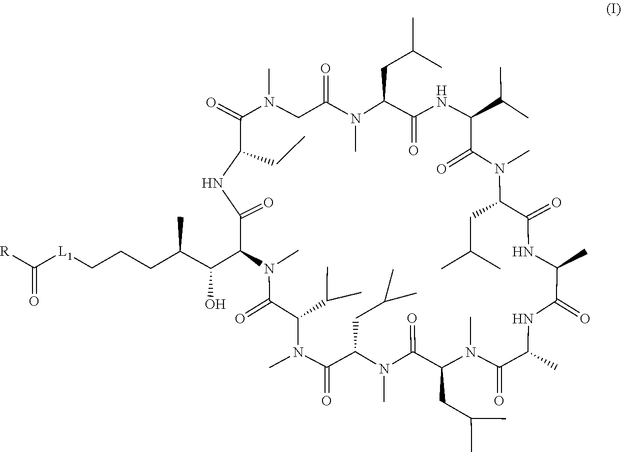
a stereoisomer thereof, or a pharmaceutically acceptable salt of any of the foregoing;
wherein
L₁ is a C₀ to C₄ alkyl group optionally substituted with one or more F;
R is selected from the group consisting of —OH and a PEG, wherein the PEG optionally comprises a linker group, L₂.

2. The method of claim 1, wherein R is selected from —OH or —NH(CH₂)₂₋₆(CH₂CH₂O)₄₂₋₄₆-O(CH₂)₀₋₅CH₃.

3. The method of claim 1, wherein R is selected from the group consisting of CH₃O—(CH₂ CH₂O)₄₄-CH₂CH₂CH₂NH—(BT051), CH₃)—(CH₂CH₂O)₄₄—CH₂CH₂NH—(BT090), and HO—(BT070).

4. The method of claim 1, wherein L₁ is an ethylene group or a propylene group.

5. The method of claim 1, wherein R is a PEG having from 40 to 50 ethylene oxide units.

6. The method of claim 1, wherein L₂ is a substituted or unsubstituted heteroalkylene group.

7. The method of claim 1 having the structure of Formula IA or a pharmaceutically acceptable salt thereof:

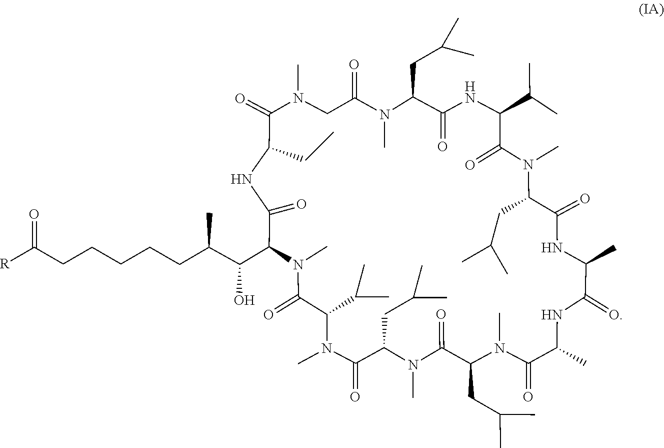

8. The method of claim 1, wherein the compound is formulated as a pharmaceutical composition comprising the compound and a pharmaceutically acceptable carrier.

9. The method of claim 1, wherein the glucose intolerance is obesity-induced.

10. The method of claim 1, wherein the glucose intolerance is associated with one or more diseases selected from the group consisting of type 2 diabetes mellitus, type 1 diabetes mellitus, gestational diabetes mellitus, neonatal diabetes, maturity-onset diabetes of the young, drug-induced diabetes, chemical-induced diabetes, hyperglycemia, impaired glucose tolerance (IGT), impaired fasting glucose (IFT), obesity, insulin resistance, metabolic syndrome, asthma, chronic obstructive pulmonary disease (COPD), Alzheimer's disease, cancers, Klinefelter Syndrome, Turner Syndrome, Wolfram Syndrome, Cystic Fibrosis, Friedreich's ataxia, multiple sclerosis, down syndrome, muscular dystrophy, polycystic ovary syndrome (PCOS), uremia, cirrhosis, chronic renal failure, and HAART-treated HIV infection.

11. The method of claim 10, wherein the disease is obesity, and wherein the obesity is diet-induced obesity.

12. The method of claim 1, wherein treatment comprises one or more of improved glucose tolerance, improved insulin sensitivity, and increased plasma glucagon-like peptide 1 (GLP-1) levels as compared to untreated control subjects.

13. The method of claim 1, wherein the method further comprises administering to the subject a therapeutically effective amount of one or more second compound that inhibits one or more of multidrug resistance protein 2 (MRP2) and HXA₃ synthase, wherein the therapeutic amount of the second compound reduces migration of neutrophils into the target tissue.

14. The method of claim 1, wherein the method further comprises administering to the subject a therapeutically effective amount of one or more compounds that increases multidrug resistance protein 1 (MRP1), wherein the therapeutic amount of the third compound reduces migration of neutrophils into the target tissue.

15. The method of claim 1, wherein said method further comprises administering one or more compounds that inhibits one or more of multidrug resistance protein 2 (MRP2) and hepoxilin A3 (HXA₃) synthase, wherein the therapeutic amount of the second compound reduces migration of neutrophils into the target tissue.

16. The method of claim 1, wherein the method further comprises administering to the subject a therapeutically effective amount of one or more compounds that increases one or more N-acylethanolamines (NAEs), wherein the therapeutic amount of the third compound reduces migration of neutrophils into the target tissue.

17. The method of claim 1, further comprising administering one or more antibiotic and/or anti-inflammatory agents selected from the group consisting of: Dalbavancin, Oritavancin, Cubicin, Tedizolid, Ceftobiprole, Ceftobiprole, Ceftolozane-tazobactam, mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, steroidal agents such as corticosteroids such as hydrocortisone, hydroxyltriamcinolone alphamethyl dexamethasone, dexamethasonephosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, non-steroidal agents such as COX inhibitors, LOX inhibitors, p38 kinase inhibitors, immunosuppresant agents such as cyclosporin, and cytokine synthesis inhibitors, tetracycline, minocycline, and doxycycline, or any combination thereof.

18. The method of claim 1, further comprising administering one or more agents selected from the group consisting of: metformin; incretin mimetics; DPP-4-resistant GLP-1 receptor agonists, such as exenatide and liraglutide, DPP-4 inhibitors (sitagliptin, vildagliptin); amylin; pramlintide acetate; oral hypoglycemic drugs; orlistat; insulin; insulin degludec; insulin detemir; human insulin; biphasic human insulin; insulin aspart; biphasic insulin aspart; glucagon-like peptide-1; semaglutide; and repaglinide; or any combination thereof.
